(19) United States
(12) Patent Application Publication
(10) Pub. No.: US 2022/0047720 A1
(43) Pub. Date: Feb. 17, 2022

(54) CONJUGATES AND NANOPARTICLES OF HYALURONIC ACID AND EPIGALLOCATECHIN-3-O-GALLATE AND USES THEREOF

(71) Applicant: Agency for Science, Technology and Research, Singapore (SG)

(72) Inventors: Motoichi Kurisawa, Singapore (SG); Kun Liang, Singapore (SG); Motomi Osato, Singapore (SG); Ki Hyun Bae, Singapore (SG); Nunnarpas Yongvongsoontorn, Singapore (SG); Joo Eun Chung, Singapore (SG); Qingfeng Chen, Singapore (SG); Fritz Lai, Singapore (SG); Zhisheng Her, Singapore (SG)

(73) Assignee: Agency for Science, Technology and Research, Singapore (SG)

(21) Appl. No.: 17/312,828

(22) PCT Filed: Dec. 11, 2019
(86) PCT No.: PCT/SG2019/050610

§ 371 Date: Jun. 10, 2021

(30) Foreign Application Priority Data
Dec. 12, 2018 (SG) 10201811120Q
Dec. 12, 2018 (SG) 10201811121W

(51) Int. Cl. A61K 47/69; A61K 47/61

(52) U.S. Cl. CPC A61K 47/6939; B82Y 5/00; A61K 47/61

(57) ABSTRACT

Disclosed herein is a nanoparticle composition comprising nanoparticles formed from one of: a conjugate of dimeric epigallocatechin-3-O-gallate and hyaluronic acid; a conjugate of epigallocatechin-3-O-gallate and hyaluronic acid; or a epigallocate-chin-3-O-gallate-terminated hyaluronic acid conjugate; and an active agent or a pharmaceutically acceptable salt, solvate or prodrug 0thereof suitable to treat acute myeloid leukaemia, wherein the active agent is encapsulated in the nanoparticles.

### FIELD OF INVENTION

The invention relates to a nanoparticle composition comprising a conjugate of hyaluronic acid and epigallocatechin-3-O-gallate, and an active agent, and the use of said conjugate and nanoparticle composition for treating acute myeloid leukemia.

### BACKGROUND

Acute myeloid leukemia (AML) has become a significant global health problem, accounting for an estimated 1,000,000 new cases and 147,100 deaths annually in the world (Lancet. 2016, 388, 1545-1602). AML is a biologically complex and heterogeneous blood cancer, characterised by the infiltration of bone marrow, blood, and other tissues by malignant cells of the myeloid lineage (myeloid blast cells). Such cells suffer from blockage in the differentiation pathways, which leads to the crowding out of normal blood cells and platelets. The stages in which differentiation is arrested define the subtypes of AML (AML-M0 to M7). In spite of progress in diagnosis and therapeutic strategies, the overall 5-year survival rate is only 25% in the US and 15-20% in Europe. A more serious problem is that the relapse rates still remain high at 40% in patients younger than 60 years and 10-20% of patients above 60 years (J. Kell, Leuk Res. 2016, 47, 149-60; Betul Oran, Daniel J. Weisdorf, Haematologica 2012, 97, 1916-1924).

The first-line treatment of AML primarily involves chemotherapy and is classified in two phases: (i) remission induction phase aiming to lower the number of leukemic blasts and (ii) post-remission phase aiming to prevent disease recurrence. The standard treatment during the remission induction phase is mainly based on combination chemotherapy with cytarabine (ara-C) and an anthracycline (e.g., daunorubicin, doxorubicin, idarubicin) (H. Dombret, C. Gardin, Blood 2016, 127, 53-61). Although complete remission is achieved in nearly 80% of patients, such chemotherapeutic drugs cause severe and sometimes life-threatening side effects, including myelosuppression, gastrointestinal toxicity, and cerebral toxicity because they can damage healthy tissues and organs as a result of their non-specific mode of action. The treatment for the post-remission phase usually involves multiple cycles of high-dose chemotherapy using cytarabine (with or without radiation therapy) and stem cell transplantation (R. M. Stone, Semin Hematol. 2001, 38, 17-23). Despite the effectiveness of stem cell transplantation in reducing the risk of relapse, it is complicated and can be fatal for older and/or fragile patients who may not be able to tolerate such intensive treatment.

Treatment options for patients with relapsed AML are quite limited. Allogeneic transplantation of donor stem cells is a treatment option for patients in early first relapse or second remission (F. R. Appelbaum, Leukemia 2002, 16, 157-159). Arsenic trioxide can be used for the treatment of the patients diagnosed with relapsed acute promyelocytic leukemia, a rare subtype of AML (M. S. Tallman, Best Pract. Res. Clin. Haematol. 2007, 20, 57-65). Gemtuzumab ozogamicin (Mylotarg™, Pfizer, Inc.) is a monoclonal anti-CD33 antibody conjugated to the cytotoxin, calicheamicin, and has recently been approved by the U.S. Food and Drug Administration for treatment of relapsed or refractory CD33-positive AML in adults and in pediatric patients 2 years and older (J. Kell, Expert Rev. Anticancer Ther. 2016, 16, 377-382). However, harmful side effects, including hepatotoxicity, anaphylaxis, and hemorrhage, have been reported in patients receiving gemtuzumab ozogamicin as a single agent or as part of a combination chemotherapy regimen. Therefore, there still remains a significant unmet need for effective therapeutic approaches for patients with AML.

Over the last decade, small molecule inhibitors blocking the action of certain cellular enzymes and receptors have actively been tested in clinical trials for AML. Representative examples are inhibitors of FMS-like tyrosine kinase receptor-3 (FLT3), DNA methyltransferase (DNMT), isocitrate dehydrogenase (IDH), histone deacetylase (HDAC), bromodomain and extraterminal protein (BET), disruptor of telomeric silencing 1-like (DOT1 L), lysine-specific demethylase 1 (LSD1), and the anti-apoptotic protein B-cell lymphoma 2 (BCL-2) therapies for acute myeloid leukemia (C. Saygin, H. E. Carraway, J. Hematol. Oncol. 2017, 10, 93). Among them, FLT3 inhibitors, such as midostaurin, sorafenib and sunitinib, have emerged as promising therapeutic agents for AML patients with FLT3 internal tandem duplication (FLT3-ITD) mutations. It is known that FLT3-ITD mutations are the most frequent mutations in AML, occurring in ˜23% of the patients, and associated with poor survival and increased relapse rates (M. Hassanein, et al., Clin Lymphoma Myeloma Leuk. 2016, 16, 543-549). Unfortunately, when used alone, FLT3 inhibitors induce only a transient reduction of leukemic blast cells in the circulation but not in the bone marrow, suggesting a protective role of the bone marrow niche on leukemic cells (A. Parmar, et al., Cancer Res. 2011, 71, 4696-4706; Z. Her, et al., J. Hematol. Oncol. 2017, 10, 162). Although the administration frequency and dosage of FLT3 inhibitors can be increased to achieve the ideal therapeutic drug concentrations in the bone marrow, this over-dosage can cause severe side effects, such as hepatotoxicity, leukopenia and hemorrhage, due to their accumulation in healthy tissues and non-specific inhibition of other receptor tyrosine kinases (M. I. Davis, et al., Nat. Biotechnol. 2011, 29, 1046-51).

Recently, a therapeutic regimen has been implemented for the AML-M3 subtype using both all-trans retinoic acid (ATRA) and arsenic trioxide to unblock the blast cells from differentiation arrests (D. Nowak, et al., Blood 2009, 113, 3655-3665; F. Lo-Coco, et al., N. Engl. J. Med. 2013, 369, 111-121). The differentiation therapy has transformed AML-M3 into the leukemia subtype with the best prognosis with a dramatic elevation in the 5-year survival rate of up to 85%. However, the availability of such differentiation inducing agents for AML is limited, mainly due to the lack of specificity and potency (D. Nowak, et al., Blood 2009, 113, 3655-3665; K. Petrie, et al., Curr. Opin. Hematol. 2009, 16, 84-91). Furthermore, studies citing ATRA resistance have also begun to surface, highlighting the need to consider combinatorial strategies, such as concurrent differentiation therapy and chemotherapy to enhance therapeutic efficacy (A. Tomita, et al., Int. J. Hematol. 2013, 97, 717-725; B. C. Shaffer, et al., Drug Resist. Updat 2012, 15, 62-69).

Given the above, there remains a need for new compounds or materials that demonstrate effective anti-leukemic activity, and at the same time exhibit low toxicity toward normal cells.

Epigallocatechin-3-O-gallate (EGCG) is the major constituent of green tea catechin possessing strong antioxidant, antibacterial, anti-inflammatory, and cancer preventive activities. EGCG is known to interrupt tumor progression and metastasis by modulating multiple signaling pathways essential for cancer cell survival, migration and invasion (C. S. Yang, et al., Nat. Rev. Cancer 2009, 9, 429-439; N. Khan, et al., Cancer Res. 2006, 66, 2500-2505).

### SUMMARY OF INVENTION

Surprisingly, it has been found that a conjugate of epigallocatechin-3-O-gallate and hyaluronic acid is particularly useful in treating cancer, such as acute myeloid leukaemia. Such conjugate when used alone is able to provide effective treatment of acute myeloid leukaemia with high selectivity towards cancer cells over non-cancer cells. In addition, such conjugate is able to provide a nanoparticle composition for encapsulating an active agent, which facilitates an effective, targeted delivery of the active agent to cancer cells. Advantageously, the combination of the active agent and conjugate provides a synergistic effect to the nanoparticle composition, thereby allowing effective eradication of the cancer cells with the use of a low dose of the active agent. These potentially reduces the side effect (if any) associated with the use of such active agent.

Aspects and embodiments of the invention will now be described by reference to the following numbered clauses.

1. A nanoparticle composition comprising:
nanoparticles formed from one of:
   (a) a conjugate of dimeric epigallocatechin-3-O-gallate and hyaluronic acid, where the hyaluronic acid has multiple conjugation sites in its polymer backbone, where a plurality of dimeric epigallocatechin-3-O-gallate molecules are each conjugated to one of the multiple conjugation sites in the polymer backbone of hyaluronic acid;
   (b) a conjugate of epigallocatechin-3-O-gallate and hyaluronic acid, where the hyaluronic acid has multiple conjugation sites in its polymer backbone, where a plurality of epigallocatechin-3-O-gallate molecules are each conjugated to one of the multiple conjugation sites in the polymer backbone of hyaluronic acid; or
   (c) a epigallocatechin-3-O-gallate-terminated hyaluronic acid conjugate, where an epigallocatechin-3-O-gallate molecule is covalently bonded to a terminal position of the hyaluronic acid; and
an active agent or a pharmaceutically acceptable salt, solvate or prodrug thereof suitable to treat acute myeloid leukaemia, wherein:

the active agent is encapsulated in the nanoparticles.

2. The composition according to Clause 1, wherein:

(a) the conjugate of dimeric epigallocatechin-3-O-gallate and hyaluronic acid, where the hyaluronic acid has multiple conjugation sites in its polymer backbone, where a plurality of dimeric epigallocatechin-3-O-gallate molecules are each conjugated to one of the multiple conjugation sites in the polymer backbone of hyaluronic acid may have the formula la:

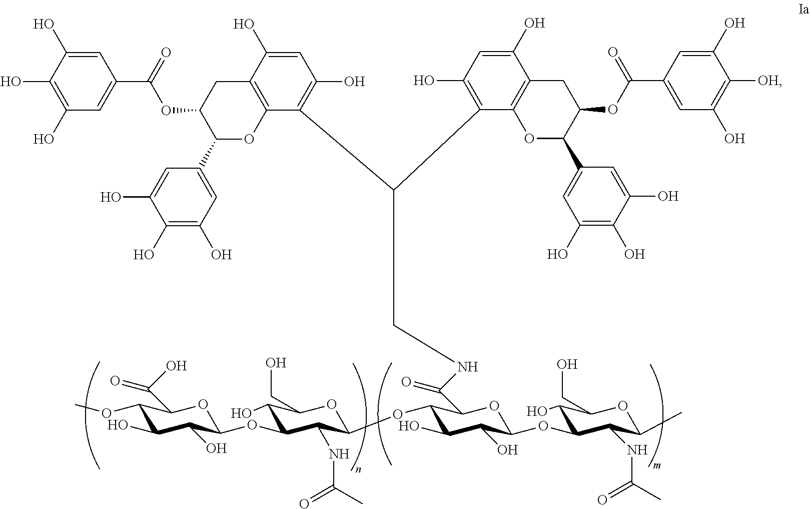

wherein each n

and m represent random repeating units in the hyaluronic acid backbone; or

(b) a conjugate of epigallocatechin-3-O-gallate and hyaluronic acid, where the hyaluronic acid has multiple conjugation sites in its polymer backbone, where a plurality of epigallocatechin-3-O-gallate molecules are each conjugated to one of the multiple conjugation sites in the polymer backbone of hyaluronic acid may have the formula Ib:

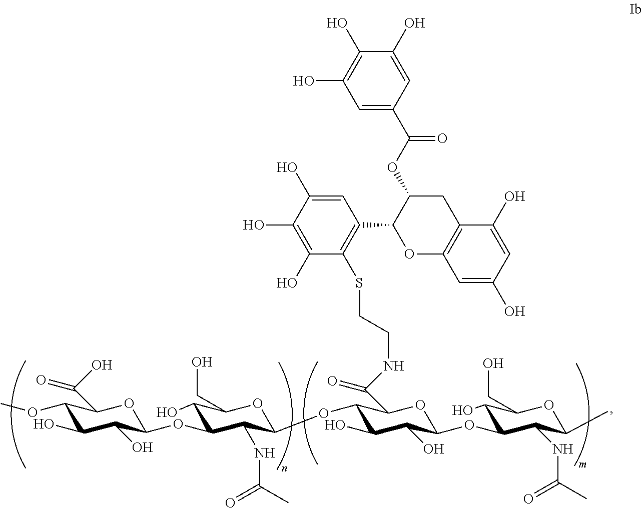

wherein each n

and m represent random repeating units in the hyaluronic acid backbone; or

(c) the epigallocatechin-3-O-gallate-terminated hyaluronic acid conjugate may have the formula Ic:

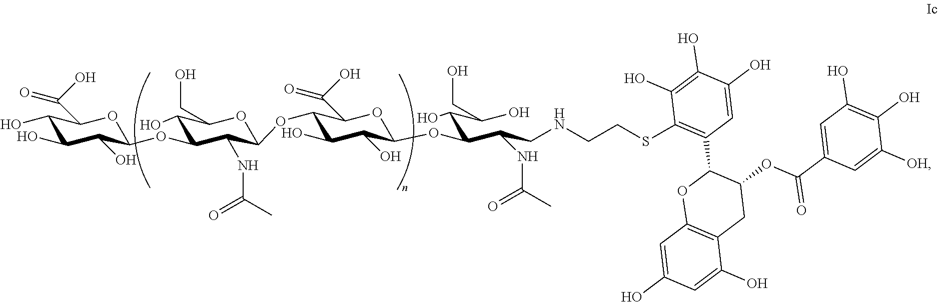

wherein n represents random repeating units in the hyaluronic acid backbone.

3. The composition according to Clause 1 or Clause 2, wherein:

(a) the epigallocatechin-3-O-gallate-terminated hyaluronic acid conjugate may have a molecular weight of from 1 to 50 kDa, such as from 10 to 30 kDa;

(b) the conjugate of the epigallocatechin-3-O-gallate and hyaluronic acid where the hyaluronic acid has multiple conjugation sites in its polymer backbone, where a plurality of epigallocatechin-3-O-gallate molecules are each conjugated to one of the multiple conjugation sites in the polymer backbone of hyaluronic acid may have a molecular weight of from 50 to 100 kDa, such as from 60 to 80 kDa; or

(c) the conjugate of dimeric epigallocatechin-3-O-gallate and hyaluronic acid where the hyaluronic acid has multiple conjugation sites in its polymer backbone, where a plurality of dimeric epigallocatechin-3-O-gallate molecules are each conjugated to one of the multiple conjugation sites in the polymer backbone of hyaluronic acid may have a molecular weight of from 50 to 100 kDa, such as from 60 to 80 kDa.

4. The composition according to any one of the preceding clauses, wherein the nanoparticle may have an average hydrodynamic diameter of from 10 to 1,000nm, such as from 90 to 500 nm, such as from 100 to 400 nm, such as from 120 to 350 nm.

5. The composition according to any one of the preceding clauses, wherein the active agent may form from 0.1 to 60 wt % of the composition, such as from 0.3 to 50 wt %, such as from 1 to 47 wt % (e.g. from 4.3 to 47 wt % or from 0.3 to 5 wt %).

6. The composition according to any one of the preceding clauses, wherein the active agent may be a FMS-like tyrosine kinase receptor-3 (FLT3) inhibitor.

7. The composition according to Clause 6, wherein the FLT3 inhibitor may be:

(a) a Type I inhibitor, optionally selected from one or more of sunitinib, lestaurtinib, midostaurin, crenolanib, and gilteritinib; or

(b) a Type II inhibitor, optionally selected from one or more of sorafenib, quizartinib, and ponatinib.

8. The composition according to Clause 7, wherein the FLT3 inhibitor may be:

(a) sunitinib; or

(b) sorafenib.

9. The composition according to any one of the preceding clauses, wherein the nanoparticles of the epigallocatechin-3-O-gallate-terminated hyaluronic acid conjugate may be core-shell nanoparticles, optionally wherein:

(ai) the core of the core-shell nanoparticles are predominantly epigallocatechin-3-O-gallate; and

(aii) the shell of the core-shell nanoparticles are predominantly hyaluronic acid.

10. A method of making a composition according to any one of Clauses 1 to 9, wherein the method comprises the steps of:

(i) adding an active agent or a pharmaceutically acceptable salt, solvate or prodrug thereof suitable to treat acute myeloid leukaemia with one of:
(a) a conjugate of dimeric epigallocatechin-3-O-gallate and hyaluronic acid, where the hyaluronic acid has multiple conjugation sites in its polymer backbone, where a plurality of dimeric epigallocatechin-3-O-gallate molecules are each conjugated to one of the multiple conjugation sites in the polymer backbone of hyaluronic acid;
(b) a conjugate of epigallocatechin-3-O-gallate and hyaluronic acid, where the hyaluronic acid has multiple conjugation sites in its polymer backbone, where a plurality of epigallocatechin-3-O-gallate molecules are each conjugated to one of the multiple conjugation sites in the polymer backbone of hyaluronic acid; or
(c) a epigallocatechin-3-O-gallate-terminated hyaluronic acid conjugate, where an epigallocatechin-3-O-gallate molecule is covalently bonded to a terminal position of the hyaluronic acid,
   in a solvent, optionally with agitation, for a period of time to provide a dispersion of nanoparticles; and
(ii) collecting the resulting nanoparticles from the dispersion of nanoparticles.

11. The method according to Clause 10, wherein:

(a) the solvent may be water (e.g. deionised water); and/or

(b) the concentration of the active agent in the solution may be from 0.001 to 1 mg mL⁻¹, such as from 0.02 to 0.8 mg mL⁻¹; and/or

(c) the concentration of the conjugate in the solution may be from 0.01 to 20 mg mL⁻¹, such as from 0.1 to 10 mg mL⁻¹.

12. A composition according to any one of Clauses 1 to 9 for use in medicine.

13. Use of a composition according to any one of Clauses 1 to 9 for the manufacture of a medicament for the treatment of acute myeloid leukaemia.

14. A composition according to any one of Clauses 1 to 9 for use in the treatment of acute myeloid leukaemia.

15. A method of treatment of acute myeloid leukaemia comprising the steps of providing a pharmaceutically effective amount of the composition according to any one of Clauses 1 to 9 to a subject in need thereof.

16. Use of a compound of formula la or Ib:

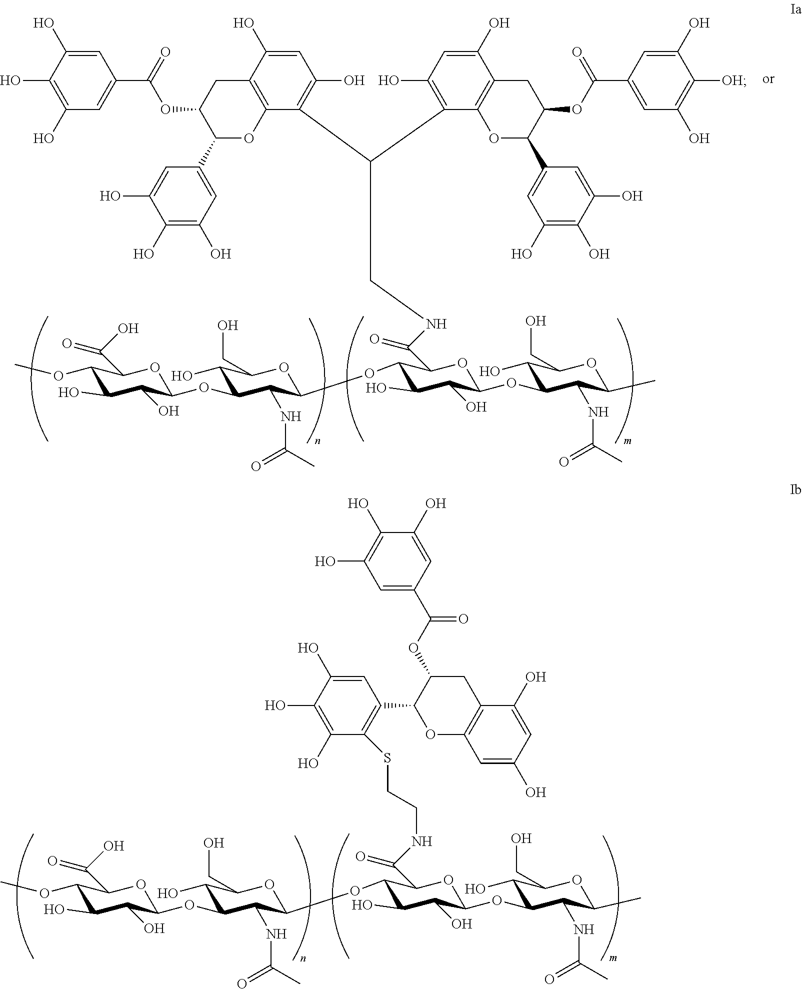

or a pharmaceutically acceptable salt, solvate or prodrug thereof for use in the preparation of a medicament to treat cancer.

17. A compound of formula la or Ib:

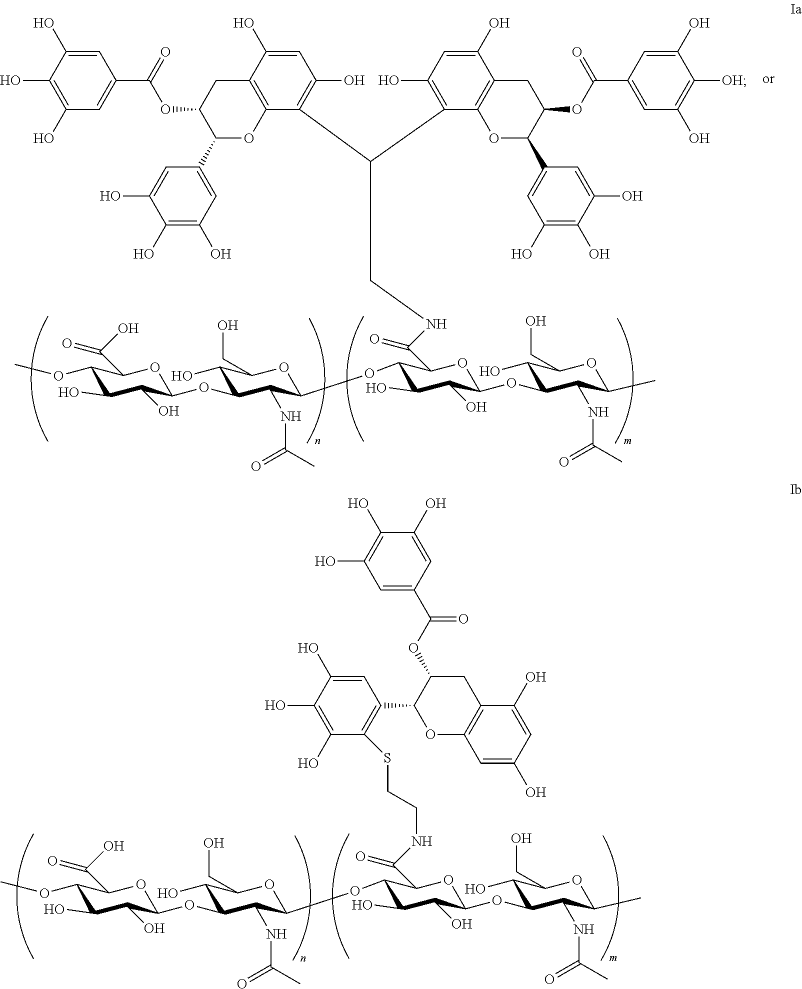

for use in the treatment of cancer.

18. A method of treatment of cancer comprising the steps of providing a pharmaceutically effective amount of a composition compound of formula la or Ib:

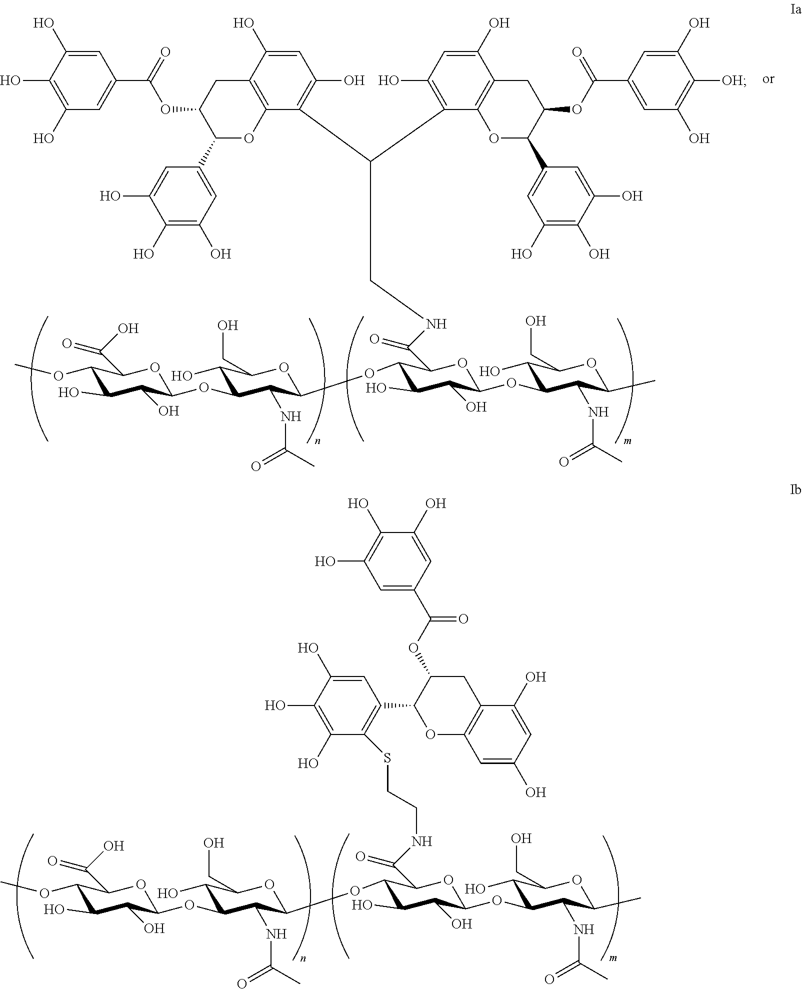

to a subject in need thereof.

19. The use, compound and method of Clauses 16, 17 and 18, respectively, wherein the cancer may be acute myeloid leukemia.

20. The use, compound and method of any one of Clauses 15 to 19, wherein:

(a) the compound of la may have a molecular weight of from 50 to 120 kDa, such as from 80 to 100 kDa; or

(b) the compound of lb may have a molecular weight of from 50 to 120 kDa, such as from 80 to 100 kDa.

### BRIEF DESCRIPTION OF DRAWINGS

FIG. 1 Depicts the chemical structures of HA-EGCG conjugates used in the current invention: (a) HA-EGCG (A) with multiple EGCG dimer molecules grafted to the HA backbone; (b) HA-EGCG (B) with multiple EGCG molecules grafted to the HA backbone; and (c) HA-EGCG (C) with a EGCG molecule conjugated to the terminal end of the HA backbone.

FIG. 2 Depicts a schematic representation of the strategy of the current invention to self-assemble HA-EGCG conjugates (20) and small inhibitor molecules (22) into nanoparticles (26) for targeted entry into leukemic blast cells (30) via the interaction of HA on the as-synthesised nanoparticles (26) with CD44 (28) on the cells.

FIG. 3 Depicts: (a) the drug loading efficiency; and (b) drug loading content of nanoparticles prepared from HA-EGCG (C) and sunitinib at varying concentrations. Results are reported as mean values (n=2).

FIG. 4 Depicts: (a) the drug loading efficiency; and (b) drug loading content of nanoparticles prepared from HA-EGCG (C) and sorafenib at varying concentrations. Results are reported as mean values (n=2).

FIG. 5 Depicts the in vitro anti-leukemic activity of HA-EGCG/sunitinib nanoparticles (Suni-NP-1, Suni-NP-2, Suni-NP-3 and Suni-NP-4) and free sunitinib on: (a) MOLM-14 cells; and (b) MV-4-11 cells. Data are presented as mean±standard deviation (n=4).

FIG. 6 Depicts the in vitro anti-leukemic activity of HA-EGCG/sorafenib nanoparticles (Sora-NP-1, Sora-NP-2, Sora-NP-3 and Sora-NP-4) and free sorafenib on: (a) MOLM-14 cells; and (b) MV-4-11 cells. Data are presented as mean±standard deviation (n=4).

FIG. 7 Depicts the in vitro anti-leukemic activity of Suni-NP-1, Sora-NP-1, HA-EGCG (C) conjugate and free EGCG on MOLM-14 and MV-4-11 cells, respectively, as a function of EGCG unit concentration. In this study, HA-EGCG (C) was selected as the HA-EGCG conjugate because it was used to produce Suni-NP-1 and Sora-NP-1. Data are presented as mean±standard deviation (n=4).

FIG. 8 Depicts: (a) initial; and (b) subsequent, consolidated results of the time-course changes of the proportion of human CD45⁺ cells in the peripheral blood of Leu 14-engrafted NSG mice that received intravenous injection of free sorafenib or Sora-NP-1 at a sorafenib dose of 0.4 mg kg⁻¹ twice weekly for 4 weeks. Data are presented as mean percentages of human CD45⁺ cells relative to total cells±standard deviation (n=2−5 for initial results; n=3−8 for subsequent results). For figure (a), asterisks indicate a statistically significant difference between two groups; *P<0.05; ***P<0.0005; n.s.: non-significant. For FIG. (b), asterisks indicate a statistically significant difference versus the control group; *P<0.05; ***P<0.0005.

FIG. 9 Depicts: (a) initial; and (b) subsequent, consolidated results of the proportion of human CD45⁺ cells in the spleen and bone marrow of Leu 14-engrafted NSG mice harvested at the end of 4-week treatments of free sorafenib or Sora-NP-1 at a sorafenib dose of 0.4 mg kg⁻¹. One mouse in the free sorafenib-treated group died 25 days after the first treatment was excluded from the endpoint analysis. Data are presented as mean percentages of human CD45⁺ cells relative to total cells±standard deviation (n=1−5 for initial results; n=3−8 for subsequent results). Asterisks indicate a statistically significant difference between two groups; **P<0.005; ***P<0.0005; n.s.: non-significant.

FIG. 10 Depicts a Kaplan-Meier plot of survival probability for Leu 14-engrafted NSG mice receiving 4-week treatments of free sorafenib or Sora-NP-1 at a sorafenib dose of 0.4 mg kg⁻¹. Asterisks indicate a statistically significant difference versus the control group; **P<0.005; ***P<0.0005.

FIG. 11 Depicts a schematic representation of the use of HA-EGCG (A) and (B) conjugates (40) in selectively targeting of AML cells 46 (i.e. myeloid blast cells) via HA binding to CD44 receptors overexpressed on the cell surface. Upon internalisation, HA-EGCG conjugates can achieve anti-leukemic activity by a combination of two effects—elimination (42) of the blast cells by triggering cell death (48) of the blast cells, or induction of terminal differentiation (44) of the cells into monocytes (50) or granulocytes (52).

FIG. 12 Depicts the flow cytometric profiles of CD44 expression of AML cell lines (HL60 and NB4).

FIG. 13 Depicts the viability of (a) HL60; and (b) NB4 cells following treatment with HA-EGCG (A) and (B) conjugates (100 μg/mL), a physical mixture (HA+EGCG) and the individual constituents of HA and EGCG at equivalent concentrations, respectively, for 48 or 72 h. Data are presented as mean±standard deviation (n=4). ****P<0.0001 versus EGCG and HA+EGCG.

FIG. 14 Depicts: (a) the viability of AML cells (HL60 and NB4) and normal cells (HEK293 and HUVEC) when treated with various concentrations of HA-EGCG (A), and (B) conjugates and EGCG for 72 h; and (b) the viability of AML cells and normal cells when treated with HA-EGCG (A) and (B) conjugates (500 μg/mL). Data are presented as mean±standard deviation (n=4). **P<0.01; ***P<0.001; ****P<0.0001.

FIG. 15 Depicts: (a) fold-changes of CD11b, CD14 and CD15 expression in NB4 cells following treatment with HA, EGCG, HA-EGCG (A) and (B) conjugates, three well-established differentiation-inducing agents (phorbol 12-myristate 13-acetate (PMA), all-trans retinoic acid (ATRA) and anti-human CD44 antibody (A3D8)), respectively, relative to untreated control; and (b) percentage of cells expressing each of the three surface antigen markers. Data are presented as mean±standard deviation (n=3). *P<0.05; **P<0.01; ***P<0.001; ****P<0.0001 versus untreated control.

FIG. 16 Depicts: (a) fold changes of CD11b, CD14 and CD15 expression of HL60 cells following treatment with HA, EGCG, HA-EGCG (A) and (B) conjugates and three well-established differentiation-inducing agents (PMA, ATRA and A3D8), relative to untreated control; and (b) percentage of cells expressing each of the three surface antigen markers. Data are presented as mean±standard deviation (n=3). *P<0.05; **P<0.01; ***P<0.001; ****P<0.0001 versus untreated control.

FIG. 17 Depicts the flow cytometry dot-plots of non-treated HL60 cells and HL60 cells treated with HA, EGCG, HA-EGCG (A) and (B) conjugates, three well-established differentiation-inducing agents (PMA, ATRA and A3D8), respectively.

FIG. 18 Depicts the red blood cell count of HL60-xenografted mice receiving intravenous injections of HA-EGCG (B) conjugate (50 mg/kg) or PBS (control) three times a week for a total of five weeks. Data are presented as mean±standard deviation (n=5).

FIG. 19 Depicts: (a) the white blood cell count; (b) body weights; and (c) survival fraction of HL60-xenografted mice receiving intravenous injections of HA-EGCG (B) conjugate (50 mg/kg) or PBS (control) three times a week for a total of five weeks; and (d) spleen weights of the mice at the end of study, relative to those of normal, healthy mice. Data are presented as mean±standard deviation (n=5). *P<0.05, **P<0.01, ***P<0.001 versus control.

### DESCRIPTION

Thus, there is disclosed a nanoparticle composition comprising:
nanoparticles formed from one of:
   (a) a conjugate of dimeric epigallocatechin-3-O-gallate and hyaluronic acid, where the hyaluronic acid has multiple conjugation sites in its polymer backbone, where a plurality of dimeric epigallocatechin-3-O-gallate molecules are each conjugated to one of the multiple conjugation sites in the polymer backbone of hyaluronic acid;
   (b) a conjugate of epigallocatechin-3-O-gallate and hyaluronic acid, where the hyaluronic acid has multiple conjugation sites in its polymer backbone, where a plurality of epigallocatechin-3-O-gallate molecules are each conjugated to one of the multiple conjugation sites in the polymer backbone of hyaluronic acid; or
   (c) a epigallocatechin-3-O-gallate-terminated hyaluronic acid conjugate, where an epigallocatechin-3-O-gallate molecule is covalently bonded to a terminal position of the hyaluronic acid; and
an active agent or a pharmaceutically acceptable salt, solvate or prodrug thereof suitable to treat acute myeloid leukaemia, wherein: the active agent is encapsulated in the nanoparticles.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

Pharmaceutically acceptable salts that may be mentioned include acid addition salts and base addition salts. Such salts may be formed by conventional means, for example by reaction of a free acid or a free base form of a compound of an active agent suitable to treat acute myeloid leukaemia with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of formula I in the form of a salt with another counter-ion, for example using a suitable ion exchange resin.

Examples of pharmaceutically acceptable salts include acid addition salts derived from mineral acids and organic acids, and salts derived from metals such as sodium, magnesium, or preferably, potassium and calcium.

Examples of acid addition salts include acid addition salts formed with acetic, 2,2-dichloroacetic, adipic, alginic, aryl sulphonic acids (e.g. benzenesulphonic, naphthalene-2-sulphonic, naphthalene-1,5-disulphonic and p-toluenesulphonic), ascorbic (e.g. L-ascorbic), L-aspartic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, gluconic (e.g. D-gluconic), glucuronic (e.g. D-glucuronic), glutamic (e.g. L-glutamic), a-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, tartaric (e.g.(+)-L-tartaric), thiocyanic, undecylenic and valeric acids.

Particular examples of salts are salts derived from mineral acids such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

As mentioned above, also encompassed by an active agent suitable to treat acute myeloid leukaemia are any solvates of said compounds and their salts. Preferred solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulphoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGA), differential scanning calorimetry (DSC) and X-ray crystallography.

The solvates can be stoichiometric or non-stoichiometric solvates. Particularly preferred solvates are hydrates, and examples of hydrates include hemihydrates, monohydrates and dihydrates.

For a more detailed discussion of solvates and the methods used to make and characterise 0them, see Bryn et al., Solid-State Chemistry of Drugs, Second Edition, published by SSCI, Inc of West Lafayette, Ind., USA, 1999, ISBN 0-967-06710-3.

The term “prodrug” of a relevant active agent suitable to treat acute myeloid leukaemia includes any compound that, following oral or parenteral administration, is metabolised in vivo to form that compound in an experimentally-detectable amount, and within a predetermined time (e.g. within a dosing interval of between 6 and 24 hours (i.e. once to four times daily)).

Prodrugs of an active agent suitable to treat acute myeloid leukaemia may be prepared by modifying functional groups present on the compound in such a way that the modifications are cleaved, in vivo when such prodrug is administered to a mammalian subject. The modifications typically are achieved by synthesizing the parent compound with a prodrug substituent. Prodrugs of active agents suitable to treat acute myeloid leukaemia include those in which a hydroxyl, amino, sulfhydryl, carboxyl or carbonyl group in the active agent is bonded to any group that may be cleaved in vivo to regenerate the free hydroxyl, amino, sulfhydryl, carboxyl or carbonyl group, respectively.

Examples of prodrugs include, but are not limited to, esters and carbamates of hydroxyl functional groups, esters groups of carboxyl functional groups, N-acyl derivatives and N-Mannich bases. General information on prodrugs may be found e.g. in Bundegaard, H. “Design of Prodrugs” p. 1-92, Elsevier, New York-Oxford (1985).

When used herein, the term “nanoparticle” is intended to refer to particles that have an average hydrodynamic diameter of from 0.1 to 2,000 nm. In more particle embodiments of the invention that may be disclosed herein, the nanoparticles may have an average hydrodynamic diameter of from 1 to 1,000 nm, such as from 100 to 400 nm, such as from 120 to 350 nm. For the avoidance of doubt, it is explicitly contemplated that where a number of numerical ranges related to the same feature are cited herein, that the end points for each range are intended to be combined in any order to provide further contemplated (and implicitly disclosed) ranges. Thus, in relation to the above related numerical ranges, there is disclosed:
0.1 to 1 nm, 0.1 to 100 nm, 0.1 to 120 nm, 0.1 to 350 nm, 0.1 to 400 nm, 0.1 to 1,000 nm, 0.1 to 2,000 nm;
1 to 100 nm, 1 to 120 nm, 1 to 350 nm, 1 to 400 nm, 1 to 1,000 nm, 1 to 2,000 nm;
100 to 120 nm, 100 to 350 nm, 100 to 400 nm, 100 to 1,000 nm, 100 to 2,000 nm;
120 to 350 nm, 120 to 400 nm, 120 to 1,000 nm, 120 to 2,000 nm;
350 to 400 nm, 350 to 1,000 nm, 350 to 2,000 nm;
400 to 1,000 nm, 400 to 2,000 nm; and
1,000 to 2,000 nm.

The conjugates of the current invention may have any suitable molecular weight. Examples of suitable molecular weights include from 1 to 1,500 kDa, such as from 2 to 1,000 kDa, such as from 25 to 150 kDa, such as from 50 to 100 kDa, such as from 60 to 80 kDa, such as from 2 to 50 kDa, such as from 10 to 30 kDa.

When used herein, the term “conjugate of dimeric epigallocatechin-3-O-gallate and hyaluronic acid” refers to a material formed by covalently bonding each one of a plurality of dimeric epigallocatechin-3-O-gallate molecules to a suitable conjugation site (i.e. a functional group capable of forming a covalent bond to the dimeric epigallocatechin-3-O-gallate) on the polymer backbone of hyaluronic acid. In certain embodiments that may be described herein, the conjugate of dimeric epigallocatechin-3-O-gallate and hyaluronic acid, where the hyaluronic acid has multiple conjugation sites in its polymer backbone, where a plurality of dimeric epigallocatechin-3-O-gallate molecules are each conjugated to one of the multiple conjugation sites in the polymer backbone of hyaluronic acid may have the formula la:

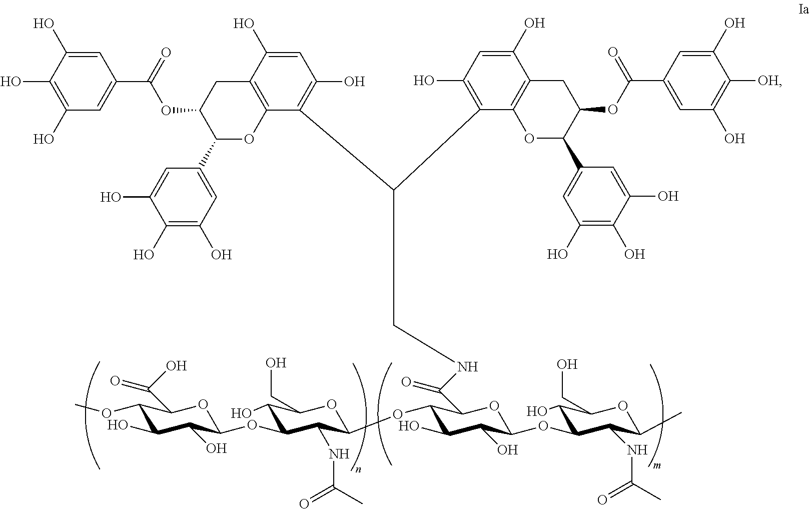

wherein each n and m represent random repeating units in the hyaluronic acid backbone. Any suitable molecular weight of the conjugate of dimeric epigallocatechin-3-O-gallate and hyaluronic acid may be used in embodiments of the invention. For example, the conjugate of dimeric epigallocatechin-3-O-gallate may have a molecular weight of from 50 to 100 kDa, such as from 60 to 80 kDa.

When used herein, the term “conjugate of epigallocatechin-3-O-gallate and hyaluronic acid” refers to a material formed by covalently bonding each one of a plurality of epigallocatechin-3-O-gallate molecules (i.e. non-dimeric molecules) to a suitable conjugation site (i.e. a functional group capable of forming a covalent bond to the epigallocatechin-3-O-gallate) on the polymer backbone of hyaluronic acid. In certain embodiments that may be described herein, the conjugate of epigallocatechin-3-O-gallate and hyaluronic acid, where the hyaluronic acid has multiple conjugation sites in its polymer backbone, where a plurality of epigallocatechin-3-O-gallate molecules are each conjugated to one of the multiple conjugation sites in the polymer backbone of hyaluronic acid may have the formula Ib:

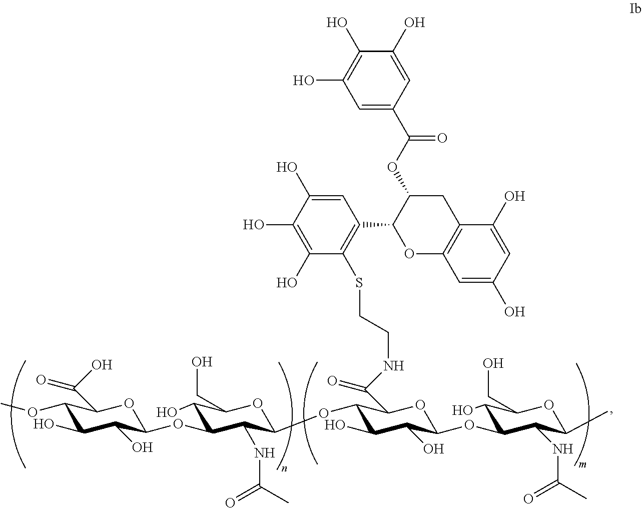

wherein each n and m represent random repeating units in the hyaluronic acid backbone. Any suitable molecular weight of the conjugate of epigallocatechin-3-O-gallate and hyaluronic acid may be used in embodiments of the invention. For example, the conjugate of epigallocatechin-3-O-gallate and hyaluronic acid may have a molecular weight of from 50 to 100 kDa, such as from 60 to 80 kDa.

When used herein, the term “epigallocatechin-3-O-gallate-terminated hyaluronic acid conjugate” refers to a material formed by covalently bonding one epigallocatechin-3-O-gallate molecule (e.g. a non-dimeric molecule) to a terminal position of the hyaluronic acid. The terminal position of the hyaluronic acid may be the result of a ring-opening reaction between the sugar hemi-acetal and a suitable functional group attached to epigallocatechin-3-O-gallate. In certain embodiments that may be described herein, the epigallocatechin-3-O-gallate-terminated hyaluronic acid conjugate may have the formula Ic:

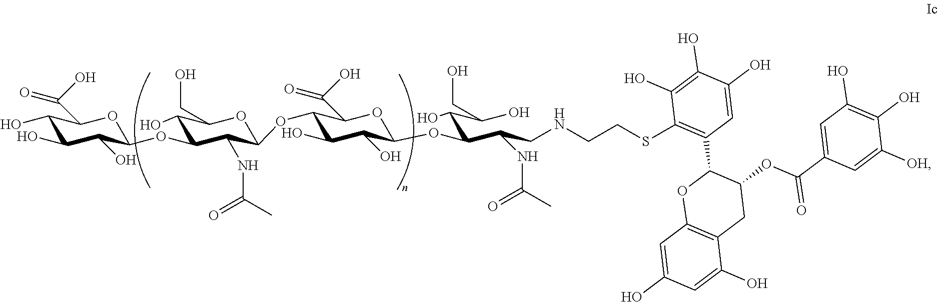

wherein n represents random repeating units in the hyaluronic acid backbone. Any suitable molecular weight of the conjugate of epigallocatechin-3-O-gallate-terminated hyaluronic acid may be used in embodiments of the invention. For example, the conjugate of epigallocatechin-3-O-gallate-terminated hyaluronic acid may have a molecular weight of from 1 to 50 kDa, such as from 10 to 30 kDa.

In any of the compositions discussed above, the active agent (i.e. the active agent suitable to treat acute myeloid leukaemia) may be present in any suitable amount of said composition. For example, the active agent may be present in an amount of from 0.00001 to 99 wt % of the composition, such as from 0.1 to 60 wt % of the composition, such as from 0.3 to 50 wt %, such as from 1 to 47 wt % (e.g. from 4.3 to 47 wt % or from 0.3 to 5 wt %).

When used herein, the terms “active agent” and “active agent suitable to treat acute myeloid leukaemia” are intended herein to refer to a material (other than the conjugate nanoparticles) that can be used to treat acute myeloid leukaemia. Examples of suitable active agents include, but are not limited to, FMS-like tyrosine kinase receptor-3 (FLT3) inhibitors, type I FLT3 inhibitors and/or type II FLT3 inhibitors. Examples of type I FLT3 inhibitors include, but are not limited to sunitinib, lestaurtinib, midostaurin, crenolanib, and gilteritinib. Examples of type I FLT3 inhibitors include, but are not limited to sorafenib, quizartinib, and ponatinib. For the avoidance of doubt, any reference herein to an active agent is intended to also include pharmaceutically acceptable salt, solvate or prodrugs thereof.

While the nanoparticles of the conjugate materials described above may take any nanoparticulate form, they may be discussed in particular embodiments described herein as core-shell nanoparticles. In particular examples that may be mentioned herein, the epigallocatechin-3-O-gallate-terminated hyaluronic acid conjugate may be provided as core-shell nanoparticles. When the conjugates provide a core-shell nanoparticle structure, the core portion of the core-shell nanoparticles may be predominantly epigallocatechin-3-O-gallate, and the shell of the core-shell nanoparticles may predominantly be hyaluronic acid (of the conjugate). In other words, the conjugate may self-assemble to provide the epigallocatechin-3-O-gallate in the core portion of the core-shell nanoparticle, with the hyaluronic acid forming the shell portion. As will be appreciated, as this core-shell nanoparticles form by self-assembly there may remain an amount of the other material in the core and/or the shell. Thus, when used herein, the term “predominantly” is intended to mean that the majority (i.e. greater than 50%) of the material in the core or shell is the predominant material. That is, the core may be formed from 55 to 100 wt % of the epigallocatechin-3-O-gallate portion of the conjugate material, such as from 60 to 99 wt %, such as from 70 to 95 wt %, such as from 80 to 90 wt %. Similarly, the shell may be formed from 55 to 100 wt % of the hyaluronic acid portion of the conjugate material, such as from 60 to 99 wt %, such as from 70 to 95 wt %, such as from 80 to 90 wt %.

In a further aspect of the invention, there is provided a method of making a composition according as described above, wherein the method comprises the steps of:

(i) adding an active agent or a pharmaceutically acceptable salt, solvate or prodrug thereof suitable to treat acute myeloid leukaemia with one of:
(a) a conjugate of dimeric epigallocatechin-3-O-gallate and hyaluronic acid, where the hyaluronic acid has multiple conjugation sites in its polymer backbone, where a plurality of dimeric epigallocatechin-3-O-gallate molecules are each conjugated to one of the multiple conjugation sites in the polymer backbone of hyaluronic acid;
(b) a conjugate of epigallocatechin-3-O-gallate and hyaluronic acid, where the hyaluronic acid has multiple conjugation sites in its polymer backbone, where a plurality of epigallocatechin-3-O-gallate molecules are each conjugated to one of the multiple conjugation sites in the polymer backbone of hyaluronic acid; or
(c) a epigallocatechin-3-O-gallate-terminated hyaluronic acid conjugate, where an epigallocatechin-3-O-gallate molecule is covalently bonded to a terminal position of the hyaluronic acid,
   in a solvent, optionally with agitation, for a period of time to provide a dispersion of nanoparticles; and
(ii) collecting the resulting nanoparticles from the dispersion of nanoparticles.

In the process described above, any suitable solvent may be used. A particular solvent that may be mentioned is water (e.g. deionised water), but the solvent may also be a polar organic solvent. Examples of suitable polar organic solvents that may be used in embodiments of the invention include, but are not limited to, acetone, acetonitrile, ethanol, methanol, propanol, tetrahydrofuran, dimethyl sulfoxide and 1,4-dioxane. As will be appreciated one or more of these polar organic solvents may be used alone or in combination with water. For example, the solvent may be mixture of water and one or more organic solvents in a volume to volume ratio of from 10 to 90% water:organic solvents. Particular examples of such mixed solvent systems are described in the examples.

Any suitable concentration of the active agent in the solution may be used. For example, the concentration of the active agent in the solution may be from 0.001 to 1 mg mL⁻¹, such as from 0.02 to 0.8 mg mL⁻¹. Any suitable concentration of the active agent in the solution may be used. For example, the concentration of the conjugate in the solution may be from 0.01 to 20 mg mL⁻¹, such as from 0.1 to 10 mg mL⁻¹. These concentrations refer to the concentration achieved in step (i) of the process above.

The agitation referred to above may be conducted by any suitable means. Such as an orbital shaker, a mechanical stirrer and the like.

Any suitable period of time may be used. For example, the period of time may be from 1 second to 5 days, such as 5 seconds to 3 days. It will be appreciated that, if agitation is used, the agitation may essentially correspond to the period of time to provide the dispersion of nanoparticles. In certain embodiments mentioned herein, agitation may be an essential part of the process and the period of time where the mixtures referred to above are subject to agitation may be from 1 second to 5 days, such as 5 seconds to 3 days.

The hyaluronic acid used in the methods disclosed herein may have any suitable molecular weight. For example, the molecular weight of the hyaluronic acid may be from 1 to 1,000 kDa, such as from 2 to 1,000 kDa, such as from 50 to 100 kDa.

Unless otherwise specified herein reference to the weights of polymeric materials refers to their number average molecular weight.

As will be appreciated the compositions disclosed above may be useful in medicine and so in a further aspect of the invention, there is disclosed a composition as disclosed above for use in medicine.

Further aspects of the invention relate to:

(bi) use of a composition as disclosed above for the manufacture of a medicament for the treatment of acute myeloid leukaemia;

(bii) a composition as disclosed above for use in the treatment of acute myeloid leukaemia; and

(biii) a method of treatment of acute myeloid leukaemia comprising the steps of providing a pharmaceutically effective amount of the composition as disclosed above to a subject in need thereof.

It is also noted that the materials used to manufacture the nanoparticle portion (i.e. the conjugates) of the compositions above may also have activity against acute myeloid leukaemia. Thus, the compositions above may display a synergistic effect, through the combination of an active agent to treat acute myeloid leukaemia and the conjugate used (as shown in Examples 3 and 4 below). Given this, the use of the nanoparticle composition described herein provides an effective treatment of acute myeloid leukaemia (i.e. in targeting MOLM-14 and MV-4-11 cancer cells) via: (a) targeted delivery of the active agent to the cancer cells with the use of a conjugate of epigallocatechin-3-O-gallate and hyaluronic acid; and (b) the synergistic effect of the combination of the active agent and conjugate in killing the cancer cells. Advantageously, this allows the use of a low dose of active agents and/or conjugates for treatment, which thereby reduces the side effects (if any) to the normal cells.

Thus, in addition to the compositions above, in further aspects of the invention, there is disclosed:

(ci) use of a compound of formula la or Ib:

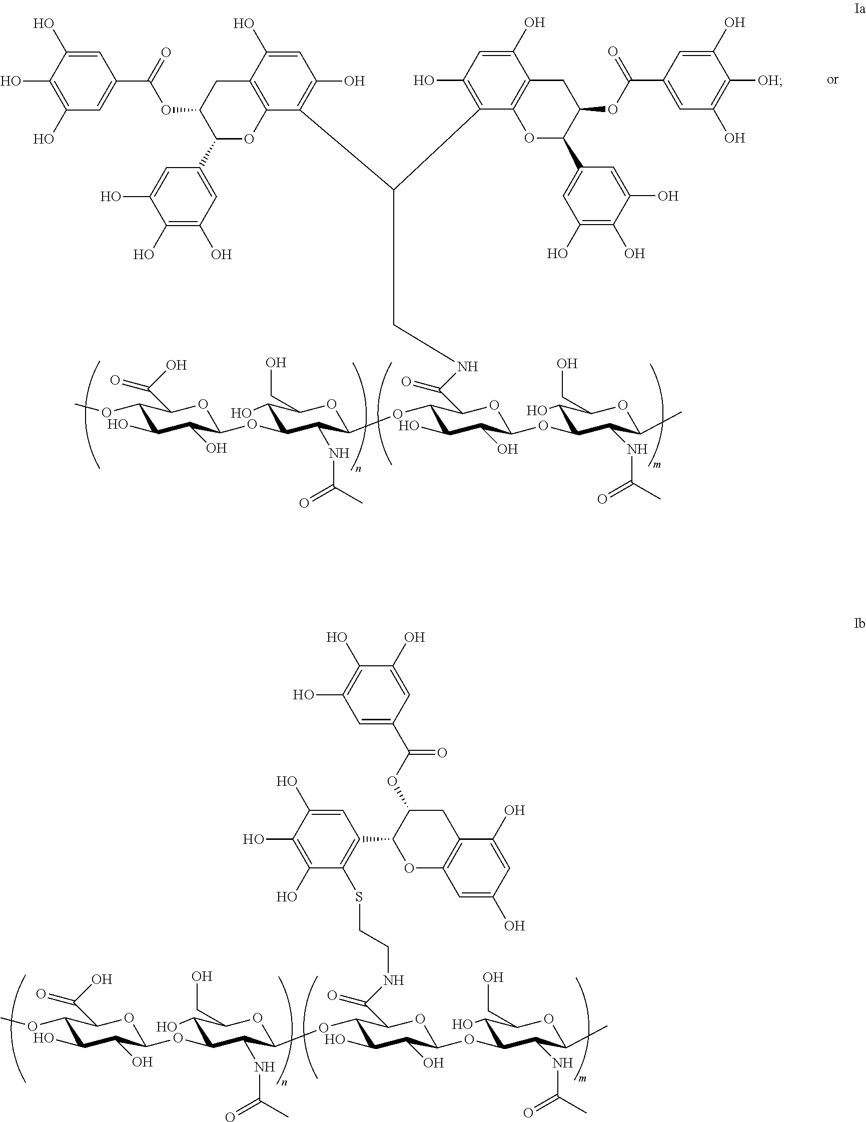

or a pharmaceutically acceptable salt, solvate or prodrug thereof for use in the preparation of a medicament to treat cancer;

(cii) a compound of formula la or Ib:

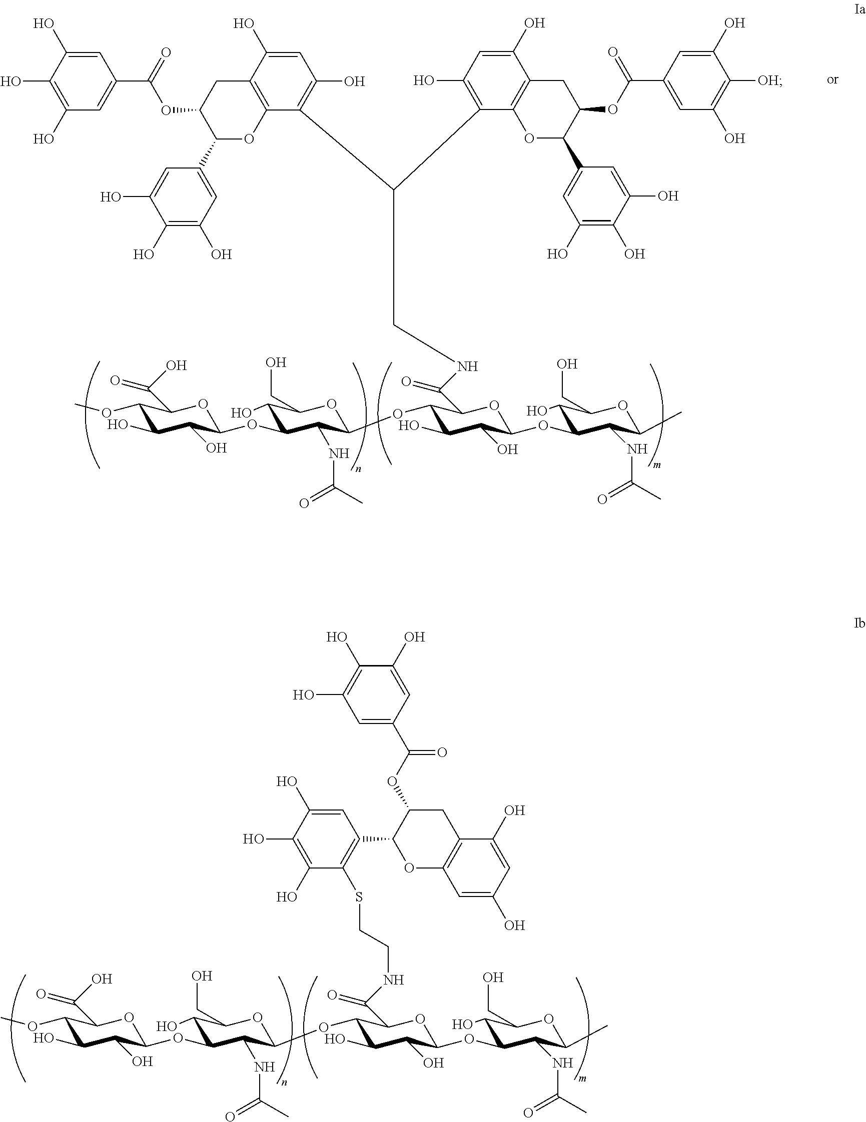

for use in the treatment of cancer; and

(ciii) a method of treatment of cancer comprising the steps of providing a pharmaceutically effective amount of a composition compound of formula la or Ib:

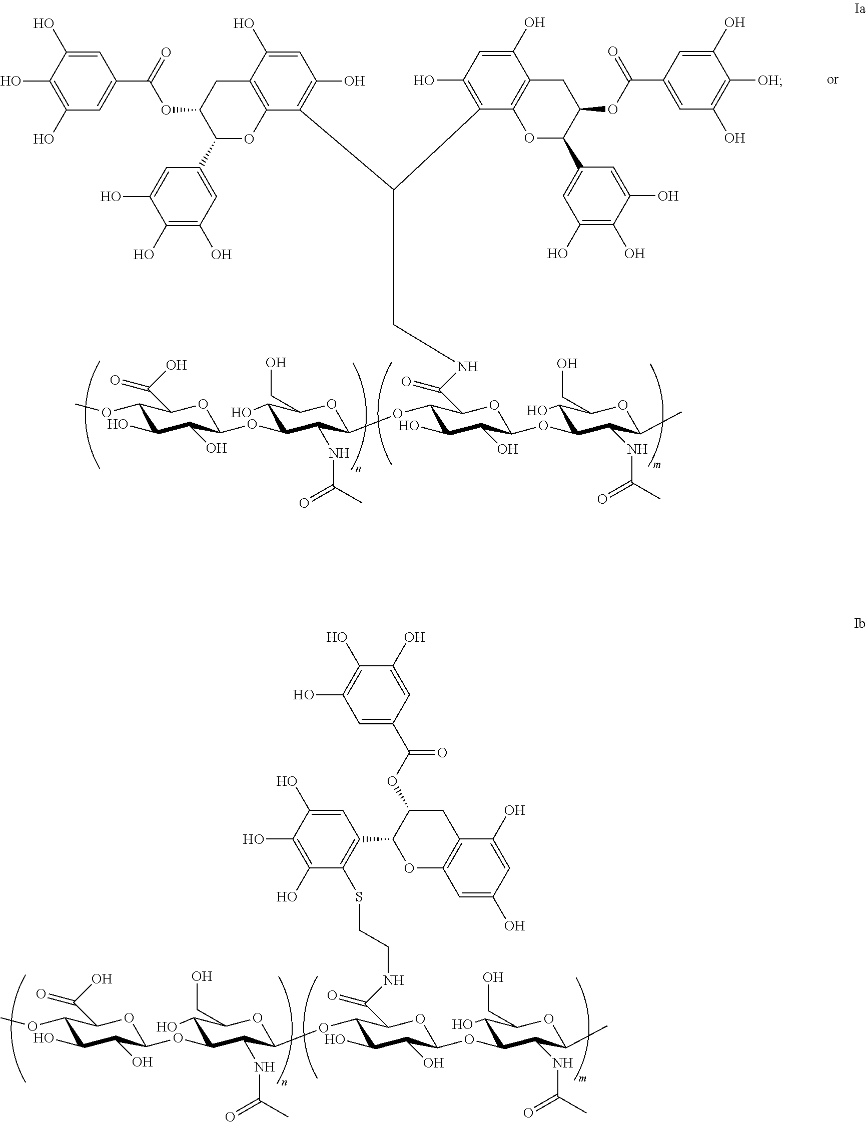

to a subject in need thereof.

The compounds disclosed herein may have any suitable molecular weight, such as from 1 to 1,500 kDa, such as from 2 to 1,000 kDa, such as from 25 to 150 kDa, such as from 20 to 120 kDa, such as from 80 to 100 kDa.

As will be appreciated, the conjugate material may be useful in the treatment of cancer more generally, but it may be particularly useful in the treatment of acute myeloid leukaemia.

The conjugate materials described for use in cancer alone are chemically identical to the conjugates described above in respect of the composition comprising a conjugate material and so reference to physical properties of the conjugate materials described hereinbefore may also apply to the materials described above. For example, the molecular weights of the conjugate materials may be the same as discussed hereinbefore. For the avoidance of doubt, the conjugate materials described in relation to the direct use in the treatment of cancer may be formulated by any suitable means known and do not need to be provided in the nanoparticulate form describe hereinbefore, though it will be appreciated that the conjugate materials described directly above can be formulated in this manner.

The conjugates alone are able to provide effective treatment of acute myeloid leukaemia with high selectivity towards cancer cells over non-cancer cells. Specifically, such conjugates show a higher toxicity towards cancer cells (i.e. HL60 and NB4 cell lines) than normal cells (i.e. HEK293 and HUVEC) as shown in Example 5. Notably, such conjugates are able to induce terminal differentiation in cancer cells, which therefore inhibits cancer progression and prolongs the survival of the cancer patients.

Further aspects and embodiments of the invention will now be described by reference to the following non-limiting examples.

### EXAMPLES

Materials

Epigallocatechin-3-gallate (EGCG, minimum 90%, TEAVIGO™) was purchased from DMS Nutritional Products Ltd. (Basel, Switzerland). Hyaluronic acid was kindly donated by JNC Corporation (Tokyo, Japan) or purchased from Lifecore Biomedical (Minnesota, USA). Sunitinib malate was a product of BioVision (Milpitas, USA). Sorafenib tosylate was obtained from AbMole BioScience (Houston, USA). Amicon Ultra-15 centrifugal filters were purchased from Merck Millipore Corporation (Darmstadt, Germany). CellTiter-Glo cell viability assay reagent (Promega Corporation, USA) was used per the manufacturer's protocol. Cystamine dihydrochloride was obtained from Merck Millipore Corporation (Darmstadt, Germany). Tris(2-carboxyethyl)phosphine hydrochloride (TCEP) and 4-(4,6-Dimethoxy-1,3,5-triazin-2-yl)-4-methylmorpholinium chloride (DMTMM) were purchased from Tokyo Chemical Industry (Tokyo, Japan). 2,2-Diethoxyethylamine (DA), N-hydroxysuccinimide (NHS), 1-ethyl-3-(3-dimethylaminopropyl)-carbodiimide hydrochloride (EDC), 2-(N-morpholino)ethanesulfonic acid (MES), all-trans retinoic acid (ATRA), phorbol 12-myristate 13-acetate (PMA) and anti-human CD44 antibody (clone: A3D8) were purchased from Sigma-Aldrich (St. Louis, USA). Mouse anti-human antibody CD44 (Bu52), isotype control antibody and fluorescein isothiocyanate (FITC)-tagged secondary antibody were acquired from Bio-Rad Laboratories (Hercules, USA). Fluorescently-labelled mouse anti-human antibodies (FITC-conjugated CD11b (ICRF-44), Cy7-conjugated CD14 (HCD14) and Cy5-conjugated CD15 (SSEA-1)) were obtained from Biolegend (San Diego, USA).

Statistical Analysis

Statistical analyses between two groups was determined by unpaired Student's t-test, while statistical differences among multiple groups were examined using one-way ANOVA and Tukey post-hoc tests, where P value smaller than 0.05 was regarded as statistically significant. Survival analysis was performed using Kaplan-Meier estimator and log-rank test was subsequently used to calculate the P value.

General Synthesis 1—HA-EGCG (A) Conjugate

HA-EGCG (A) conjugate can be synthesised by General Synthesis 1a or 1b as discussed below. Both synthesis methods will give similar HA-EGCG (A) conjugate, and HA polymers of any suitable molecular weight can be used here (e.g. 1 kDa to 1000 kDa, 76 kDa or 90 kDa).

General Synthesis 1a

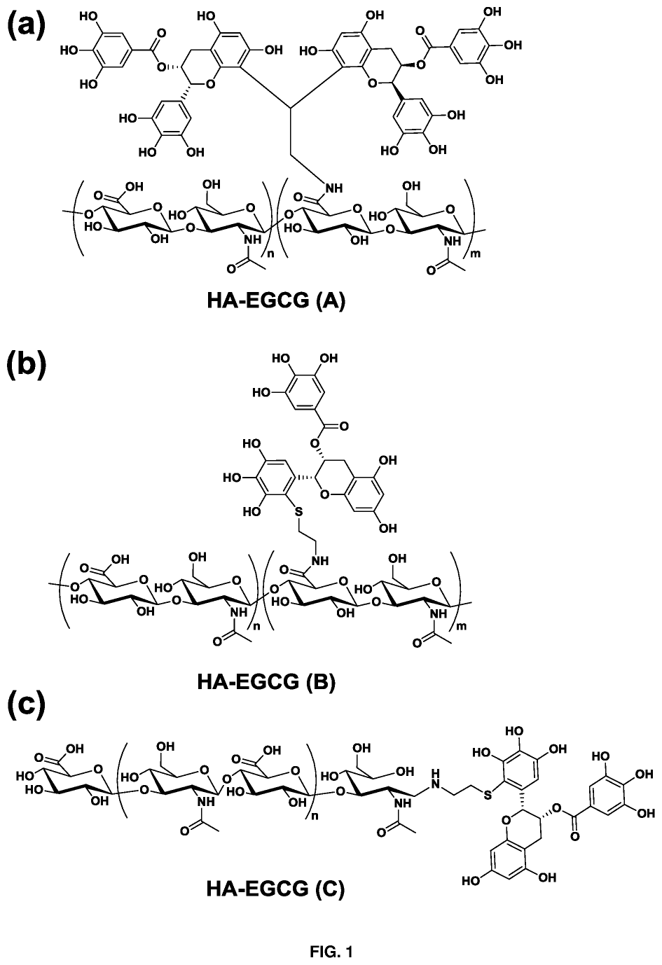

HA-EGCG (A) conjugate was synthesised by the two-step process reported in U.S. Pat. No. 8,753,687 B2. In general, the HA-EGCG (A) conjugate was synthesised via a two-step reaction, in which 2,2-diethoxyethyl amine (DA) was firstly conjugated to HA, followed by the coupling of the conjugate to EGCG. The chemical structure of HA-EGCG (A) is as shown in FIG. 1a.

In the first step of a typical reaction, HA-DA conjugates were synthesised using a standard carbodiimide coupling method with some modifications (F. Lee, et al., Soft Matter 2008, 4, 880-887). HA (5 g, 12.5 mmol of COOH) was dissolved in 500 mL of distilled water. DA (2.38 g, 17.8 mmol) was then added, followed by NHS (1.16 g, 10.0 mmol) and EDC (2.40 g, 12.5 mmol) to initiate the conjugation reaction. During the reaction, the pH of the mixture was maintained at 4.7 by the addition of 1 M NaOH. The reaction mixture was stirred overnight at room temperature and then the pH was increased to 7.0. The solution was dialysed (M_{w} cut-off: 1000 Da) against 100 mM sodium chloride solution for 2 days, 25% ethanol for 1 day and deionised water for 1 day, successively. The purified solution was lyophilised to obtain the HA-DA conjugate (about 84% yield).

In the second step, HA-DA conjugates (1 g) were first dissolved in 57 mL of deionised water. Subsequently, EGCG solution (20 equivalents of molar concentration with respect to the DA units), dissolved in 13 mL of DMSO was added. The reaction mixture was stirred under acidic condition at room temperature for 24 h. Following that, the mixture was dialysed (M_{w} cut-off: 3500 Da) against water for 3 days under nitrogen atmosphere. The purified solution was lyophilised to obtain the HA-EGCG conjugate (about 87% yield). The degree of substitution (i.e., the number of EGCG dimers per 100 disaccharide units in HA) was determined by examining the absorbance of HA-EGCG conjugates at 274 nm using a Hitachi U-2810 spectrometer. The degree of substitution for HA-EGCG (A) conjugates was determined to be 1.5.

General Synthesis 1b

Alternatively, HA-EGCG (A) conjugate can also be synthesised by the two-step process reported in F. Lee, et al., Polym Chem. 2015, 6, 462-4472. Briefly, ethylamine-bridged EGCG dimers can be synthesised first, followed by coupling of the dimers to the HA to give the desired conjugate.

In the first step, EGCG was reacted with 2,2-diethoxyethylamine (DA) to form ethylamine-bridged EGCG dimers. In brief, 145 μL of DA (1 mmol) was added to 1.2 mL of cold methanesulfonic acid (MSA):THF (1:5, v/v) while stirring. The mixture was then added to EGCG (2.29 g, 5 mmol) dissolved in 3.8 mL of THF containing 1.7 μL of MSA and stirred overnight in the dark at room temperature. The unreacted EGCG was removed by multiple extraction cycles with ethyl acetate until no free EGCG was detected. The concentration of the purified ethylamine-bridged EGCG dimer was determined by absorbance at 274 nm and was found to be 84 mg/mL (yield=88%).

In the second step, the ethylamine-bridged EGCG dimers were conjugated to HA via carbodiimide-mediated coupling reaction. In brief, HA (250 mg, 0.62 mmol) was dissolved in 19.8 mL of 0.4 M MES buffer (pH 5.2) containing 2.5 mL of DMF. NHS (89 mg, 0.78 mmol), ethylamine-bridged EGCG dimers (0.205 mmol in 2.7 mL of water) and EDC (150 mg, 0.78 mmol) were added successively and the pH of the mixture was adjusted to 4.7. The reaction mixture was purged vigorously with N₂ for 10 min and then incubated overnight under N₂. The HA-EGCG conjugates were then purified by three cycles of ethanol precipitation in the presence of NaCl. Subsequently, the precipitates were re-dissolved in 150 mL of water and dialysed against water in N₂ atmosphere overnight before lyophilisation. The final yield was 74.4%. The degree of substitution was determined by examining the absorbance of HA-EGCG conjugates at 274 nm using a Hitachi U-2810 spectrometer. The degree of substitution for HA-EGCG (A) conjugates was determined to be 0.96.

General Synthesis 2—HA-EGCG (B) Conjugate

HA-EGCG (B) conjugates were synthesised in a two-step procedure established previously in C. Liu, et al., Biomacromolecules 2017, 18, 3143-3155 and US 2016/0213787 A1. The chemical structure of HA-EGCG (B) is as shown in FIG. 1b. As will be appreciated, HA polymers of any suitable molecular weights can be used here (e.g. 1 kDa to 1000 kDa, 76 kDa or 90 kDa).

Thiolated HA derivatives were first synthesised by modifying the carboxyl groups in HA backbone with thiol groups. Typically, 1 g of HA (2.5 mmol of COOH) was dissolved in 100 mL of phosphate buffered saline (PBS) (pH 7.4). Subsequently, DMTMM (1.037 g, 3.75 mmol) and cystamine dihydrochloride (844.5 mg, 3.75 mmol) dissolved in 10 mL of PBS were added and the reaction mixture was stirred for 24 h at 25° C. The resulting solution was dialysed (M_{w} cut-off: 3500 Da) against 0.1 M NaCI solution for 2 days, 25% ethanol for 1 day and deionised water for 2 days, successively. The purified solution was lyophilised to obtain thiolated HA derivatives.

In the second step, HA-EGCG conjugates were synthesised by conjugating EGCG to the thiolated HA derivatives under mildly basic conditions. Thiolated HA derivatives (0.5 g) were dissolved in 70 mL of PBS (pH 7.4) under nitrogen-purged conditions. The solution was added dropwise to 30 ml of PBS solution containing excess of EGCG. The pH of the mixture was adjusted to 7.4 by adding 1 M NaOH and stirred for 3 h at 25° C. before adjusting to pH 6. The final mixture was dialysed (M_{w} cut-off: 3500 Da) against 25% ethanol for 1 day and deionised water for 2 days under nitrogen atmosphere. The purified solution was lyophilised to obtain HA-EGCG conjugates (about 95% yield). The degree of substitution (i.e., the number of EGCG per 100 disaccharide units in HA) was determined by examining the absorbance of HA-EGCG conjugates at 274 nm using a Hitachi U-2810 spectrometer. The degree of substitution for HA-EGCG (B) conjugates was determined to be 6.0.

General Synthesis 3—HA-EGCG (C) Conjugate

HA-EGCG (C) conjugate was synthesised in a procedure established previously in K. H. Bae, et al., Biomaterials 2017, 148, 41-53 and US 2016/0213787 A1. The chemical structure of HA-EGCG (C) is as shown in FIG. 1c. As will be appreciated, HA polymers of any suitable molecular weights can be used here (e.g. 1 kDa to 1000 kDa, or 20 kDa). The degree of substitution for HA-EGCG (C) conjugates was determined to be 0.98.

### Example 1

### Preparation of HA-EGCG Nanoparticles Containing Small Molecule Inhibitors

The HA-EGCG nanoparticles of the current invention were synthesised using one of HA-EGCG conjugates (A)-(C) with a FLT3 inhibitor. Sunitinib and sorafenib were chosen as the representative type I and type II FLT3 inhibitors respectively, for preparing these nanoparticles. Sunitinib is a type I inhibitor that blocks FLT3 signaling by binding to its intracellular ATP-binding site when the receptor is in an active conformation, while sorafenib is a type II inhibitor binding to a hydrophobic region near the ATP-binding site that is only accessible when the receptor is inactive (M. Larrosa-Garcia, M. R. Baer, Mol. Cancer Ther. 2017, 16, 991-1001).

Unlike type II inhibitors capable of killing AML cells carrying FLT3-ITD mutations only, type I inhibitors can be used to treat AML cells with FLT3-ITD mutations as well as those with FLT3 tyrosine kinase domain (TKD) mutations, which are found in ˜7% of AML patients, albeit with a more favorable prognosis than FLT3-ITD mutations (M. Hassanein, et al., Clin. Lymphoma Myeloma Leuk. 2016, 16, 543-549).

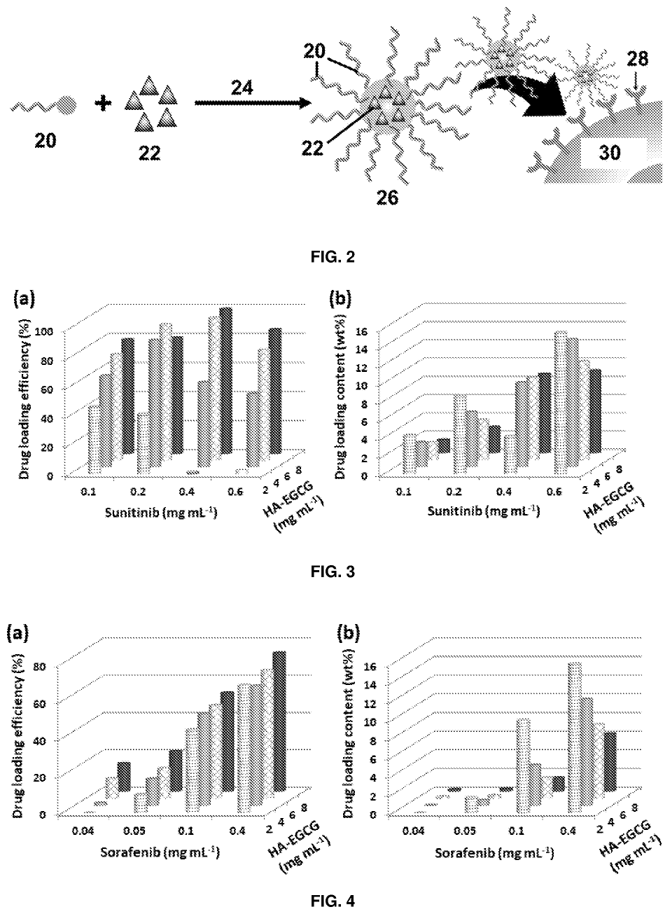

FIG. 2 shows a schematic representation of the current invention to form the self-assemble nanoparticles (26) from HA-EGCG conjugates (20) and small inhibitor molecules (22) via self-assembly and centrifugal filtration (24). The nanoparticles (26) were then used for targeted entry into leukemic blast cells (30) via the interaction of HA on the as-synthesised nanoparticles (26) with CD44 (28) on the cells.

Typically, nanoparticles comprising the HA-EGCG conjugates and sorafenib/sunitinib were prepared by mixing the HA-EGCG solution with sorafenib tosylate/sunitinib malate solution to induce the self-assembly of nanoparticles. A centrifugal filtration technique was employed to retrieve self-assembled nanoparticles, while removing unloaded polymers, drug molecules and residual solvent from the mixture.

Preparation of HA-EGCG (A) Nanoparticles Containing Sunitinib

The as-synthesised HA-EGCG (A) conjugates (from General Synthesis 1b, M_{w} of HA=76 kDa) was used in preparing the nanoparticles of the current invention. It is appreciated that HA-EGCG (A) conjugate prepared from HA with other suitable molecular weights can also be used in this preparation.

To prepare the nanoparticles, a solution of sunitinib malate (in deionsed water) was added dropwise into a solution of HA-EGCG (A) (in deionsed water) with stirring to give a final concentration of 0.2 mg min⁻¹ for each compounds. The mixture was then incubated for 1 day at 25° C. in a dark place without any agitation. The mixture was transferred to Amicon Ultra-15 centrifugal filters (M_{w} cutoff of 100 kDa) with the nanoparticles purified by centrifugation for 5 min at 2,000 ×g at 25° C. The purified nanoparticles were resuspended in 1 mL of deionised water and stored at 4° C. until use.

Preparation of HA-EGCG (B) Nanoparticles Containing Sunitinib

The as-synthesised HA-EGCG (B) conjugates (from General Synthesis 2, M_{w} of HA=76 kDa) was used in preparing the nanoparticles of the current invention. The degree of substitution (defined as the number of substituents per 100 repeating disaccharide units in HA) of the HA-EGCG (B) used in this case was determined to be 6.6. It is appreciated that HA-EGCG (B) conjugate prepared from HA with other suitable molecular weights can also be used in this preparation.

To produce the nanoparticles, a solution of sunitinib malate (in deionised water) was added dropwise into a solution of HA-EGCG (B) (in deionised water) with stirring to give a final concentration of 0.2 mg min⁻¹ for each compounds. The mixture was then incubated for 1 day at 25° C. in a dark place without any agitation. The mixture was transferred to Amicon Ultra-15 centrifugal filters (M_{w} cutoff of 100 kDa) with the nanoparticles purified by centrifugation for 5 min at 2,000 ×g at 25° C. The purified nanoparticles were resuspended in 1 mL of deionised water and stored at 4° C. until use.

Preparation of HA-EGCG (C) Nanoparticles Containing Sunitinib

The as-synthesised HA-EGCG (C) conjugates (from General Synthesis 3, M_{w} of HA=20 kDa) was used in preparing the nanoparticles of the current invention. It is appreciated that HA-EGCG (C) conjugate prepared from HA with other suitable molecular weights can also be used in this preparation.

The HA-EGCG (C) nanoparticles were prepared by mixing HA-EGCG (C) with sunitinib malate in deionised water at various concentrations. Typically, HA-EGCG (C) was vortex-mixed for 5 sec with sunitinib malate solution to give final concentrations of 2-8 mg mL⁻¹ and 0.1-0.6 mg mL⁻¹) for HA-EGCG (C) and sunitinib malate, respectively. The mixture was then incubated for 3 days at 37° C. on an orbital shaker at 50 rpm in a dark place. The mixture was transferred to Amicon Ultra-15 centrifugal filters (M_{w} cutoff of 50 kDa) with the nanoparticles purified by centrifugation for 5 min at 2,000 ×g at 25° C. The obtained nanoparticles were then further purified by dispersing in deionised water and centrifuging, for three times. The purified nanoparticles were then resuspended in 1.5 mL of deionised water and stored at 4° C. until use.

Preparation of HA-EGCG (C) Nanoparticles Containing Sorafenib

The as-synthesised HA-EGCG (C) conjugates (from General Synthesis 3, M_{w} of HA=20 kDa) was used in preparing the nanoparticles of the current invention. It is appreciated that HA-EGCG (C) conjugate prepared from HA with other suitable molecular weights can also be used in this preparation.

The HA-EGCG (C) nanoparticles were prepared by mixing HA-EGCG (C) with sorafenib tosylate in a water-solvent mixture at various concentrations. Sorafenib tosylate was dissolved in acetonitrile:methanol mixture (1:1, v/v), due to its poor water solubility. Typically, HA-EGCG (C) (in deionised water) was vortex-mixed for 5 sec with sorafenib tosylate solution to give final concentrations of 2-8 mg mL⁻¹ and 0.04-0.4 mg mL⁻¹ for HA-EGCG (C) and sorafenib tosylate, respectively. The mixture was then incubated for 2 days at 37° C. on an orbital shaker at 50 rpm in a dark place. The mixture was transferred to Amicon Ultra-15 centrifugal filters (M_{w} cutoff of 50 kDa) with the nanoparticles were retrieved by centrifugation for 5 min at 2,000 ×g at 25° C. The obtained nanoparticles were then further purified by dispersing in deionised water and centrifuging, for three times. The purified nanoparticles were resuspended in 1.5 mL of deionised water and stored at 4° C. until use.

### Example 2

### Characterisation of HA-EGCG Nanoparticles Containing Sunitinib or Sorafenib

The as-prepared HA-EGCG nanoparticles of Example 1 were characterised by dynamic light scattering to determine the hydrodynamic sizes of the particles. In addition, the drug loading efficiency and content of the nanoparticles were determined.

Experimental

Dynamic Light Scattering

The hydrodynamic diameters of the nanoparticles were examined by dynamic light scattering using a Nano ZS zetasizer (Malvern Instruments, UK). All measurements were performed at 37° C. in triplicate.

Drug Loading Efficiency and Content

To examine the loading of sunitinib in nanoparticles, each sample was diluted 50-fold in 25% ethanol-water solution and the absorbance at 431 nm was measured on a Hitachi U-2810 spectrophotometer. A calibration curve was established using various concentrations of sunitinib malate (1-10 μg mL⁻¹).

The quantity of sorafenib loaded in nanoparticles was determined by reversed-phase high-performance liquid chromatography (RP-HPLC), according to the previous report with some modifications (L. Li, et aL, J. Chromatogr. B 2010, 878, 3033-3038). Briefly, 100 μL of each sample was mixed with 500 μL of a 1:1 (v/v) mixture of acetonitrile and methanol, and then incubated for 1 h with gentle shaking to extract sorafenib from the nanoparticles. After centrifugation for 8 min at 10,000 g at 4° C., the amount of sorafenib in the supernatant was analysed using a Waters 2695 separation module equipped with a Discovery HS C18 column (5 μm, 4.6 mm i.d. ×250 mm, Supelco). The samples were eluted with acetonitrile-water mixture (65:35, v/v) at a flow rate of 1 mL min⁻¹ at 25° C. The elution of sorafenib was monitored at 265 nm and analysed using Empower 3 chromatography data software (Waters Corporation, USA). A calibration curve was established using a series of sorafenib tosylate solution (0.4-50 μg mL⁻¹). The weight of the freeze-dried nanoparticles was also measured. The drug loading content and loading efficiency were calculated by the following equations:

$$\begin{matrix}Drug loading content ( \% ) = \frac{Weight of the drug in nanoparticles}{Total weight of nanoparticles} x 100 \% & Eqn . ( 1 ) \\ Drug loading efficiency ( \% ) = \frac{Weight of the drug in nanoparticles}{Weight of the drug added} x 100 \% & Eqn . ( 2 )\end{matrix}$$

Results and Discussion

The drug loading capacity of nanoparticles comprising HA-EGCG (C) and sunitinib were first examined. It was observed that the drug loading efficiency (FIG. 3a) and loading content (FIG. 3b) of the nanoparticles could be modulated by varying the concentrations of HA-EGCG (C) and sunitinib used in the formulations. Generally, raising the concentration of HA-EGCG (C) led to increased loading efficiency with a concomitant decrease in the drug content, suggesting that greater interactions between EGCG and sunitinib occurred at higher concentrations of HA-EGCG (C). Sunitinib loading efficiency was significantly increased up to over 99%, when concentrations of HA-EGCG (C) and sunitinib were 6 and 0.4 mg mL⁻¹, respectively. Dynamic light scattering experiments were also conducted to examine hydrodynamic diameters of the nanoparticles which are as shown in Table 1.

TABLE 1
Characteristics of HA-EGCG/sunitinib nanoparticles selected for in vitro studies

| | | Initial HA- | Initial | | Drug | Drug |
|---|---|---|---|---|---|---|
| | | EGCG | sunitinib | Average | loading | loading |
| Sample | Type of | concentration | concentration | hydrodynamic | efficiency | content |
| code | HA-EGCG | (mg mL⁻¹) | (mg mL⁻¹) | diameter (nm) | (%) | (wt%) |
| Suni-NP-1 | HA-EGCG (C) | 6 | 0.2 | 95.9* | 92.5 ± 1.06 | 4.36 ± 0.09 |
| Suni-NP-2 | HA-EGCG (C) | 6 | 0.4 | 164.8* | 98.8 ± 0.82 | 8.89 ± 0.19 |
| Suni-NP-3 | HA-EGCG (B) | 0.2 | 0.2 | 177.9* | 56.1 ± 1.33 | 35.9 ± 0.57 |
| Suni-NP-4 | HA-EGCG (A) | 0.2 | 0.2 | 142.3* | 88.4 ± 1.58 | 46.9 ± 0.47 |

*Denotes intensity-weighted average diameters.

On the basis of the drug loading capacity and particle size, two compositions were selected for in vitro anti-leukemic activity studies (in Example 3) and named as Suni-NP-1 and Suni-NP-2 (Table 1). In addition, HA-EGCG (B)/sunitinib and HA-EGCG (A)/sunitinib nanoparticles were screened in a similar manner and named as Suni-NP-3 and Suni-NP-4, respectively. Notably, all HA-EGCG/sunitinib nanoparticles were produced in the 95-180 nm size range. Their nanometer dimensions are desirable to achieve long circulation and preferential tumor extravasation via the enhanced permeability and retention (EPR) effect, a phenomenon by which nanomaterials tend to pass through the leaky tumor blood vessels and reside in the tumor for extended periods of time due to impairment of lymphatic drainage (H. Maeda, et al., Adv Drug Deliv. Rev. 2013, 65, 71-79).

It was also observed that sunitinib loading content of Suni-NP-3 and Suni-NP-4 (35.9-46.9 wt %) was markedly higher than those of Suni-NP-1 and Suni-NP-2 (4.4-8.9 wt %), as well as other previously reported nanoformulations based on poly(ethylene glycol)-poly(lactic-co-glycolic acid) (PEG-PLGA) micelles (0.8-5.1 wt %) (M. Huo, et al., J. Control Release 2017, 245, 81-94). This was probably due to the hydrogen-bonding, hydrophobic and ττ-ττ stacking interactions between sunitinib and EGCG moieties which contributed to the efficient incorporation of sunitinib in HA-EGCG-based nanoparticles. Further, it was inferred that HA-EGCG conjugates having multiple EGCG moieties along the HA backbone might bind to sunitinib more efficiently than those having a single EGCG molecule per HA chain, affording a more efficient encapsulation of sunitinib.

For the HA-EGCG (C)/sorafenib conjugates, it was observed that the concentrations of the HA-EGCG (C) and sorafenib had a significant influence on the drug loading efficiency (FIG. 4a) and loading content (FIG. 4b) of the nanoparticles. Similar to sunitinib-loaded nanoparticles, the loading efficiency of sorafenib-loaded nanoparticles gradually increased with raising the concentration of EGCG-terminated HA, with an accompanying decline in the drug content. Sorafenib loading efficiency was increased up to 74%, when concentrations of HA-EGCG and sorafenib were 8 and 0.4 mg mL⁻¹, respectively. The presence of EGCG-enriched core probably contributed to the efficient encapsulation of sorafenib in the nanoparticles. The hydrodynamic diameters of the nanoparticles were also examined by dynamic light scattering. No particle structure was observed upon mixing unmodified HA with sorafenib under the conditions used to make the nanoparticles, suggesting that the existence of EGCG moieties plays an important role in the nanoparticle self-assembly.

Based on the drug loading capacity and particle size, three compositions were selected for in vitro anti-leukemic activity studies (in Example 3) and were named as Sora-NP-1 to Sora-NP-3 (Table 2). Among them, Sora-NP-1 had the smallest particle size with the lowest sorafenib loading capacity. Notably, all Sora-NP compositions gave a transparent solution without any precipitates, while free sorafenib suspended in water at the same concentration was heavily flocculated and eventually precipitated. This provides indirect evidence that sorafenib molecules were stably encapsulated in the interior of Sora-NP. The excellent dispersion stability of Sora-NP compositions would be beneficial for their clinical applications.

TABLE 2
Characteristics of HA-EGCG/sorafenib nanoparticles selected for in vitro studies

| | | Initial HA- | Initial | | Drug | Drug |
|---|---|---|---|---|---|---|
| | | EGCG | sorafenib | Average | loading | loading |
| Sample | Type of | concentration | concentration | hydrodynamic | efficiency | content |
| code | HA-EGCG | (mg mL⁻¹) | (mg mL⁻¹) | diameter (nm) | (%) | (wt%) |
| Sora-NP-1 | HA-EGCG (C) | 8 | 0.05 | 266.5* | 21.4 ± 1.02 | 0.32 ± 0.03 |
| | | | | 122.4\hat{ } | | |
| Sora-NP-2 | HA-EGCG (C) | 8 | 0.1 | 349.6* | 52.8 ± 0.05 | 1.52 ± 0.05 |
| | | | | 190.1\hat{ } | | |
| Sora-NP-3 | HA-EGCG (C) | 4 | 0.1 | 359.8* | 49.3 ± 3.13 | 4.27 ± 0.10 |
| | | | | 342.0\hat{ } | | |

*Denotes intensity-weighted average diameters.
\hat{ }Denotes number-weighted average diameters.

### Example 3

### In Vitro Anti-Leukemic Activity of as-Prepared HA-EGCG Nanoparticles Containing Sunitinib or Sorafenib

The in vitro anti-leukemic activity of selected HA-EGCG nanoparticles containing sunitinib or sorafenib (in Example 2) were evaluated on MOLM-14 and MV-4-11 cells.

Experimental

MOLM-14 and MV-4-11 cells (ATCC, USA) were maintained in RPMI 1640 media supplemented with 10% (v/v) fetal bovine serum (FBS) and 1% (v/v) penicillin/streptomycin. The cells seeded on white-walled 96-well plates (1×10⁴ cells per well) were incubated in 100 μL of 10% FBS-supplemented media containing either sunitinib- or sorafenib-loaded nanoparticles and the respective free drugs at various concentrations. In the case of free sorafenib, a stock solution of sorafenib tosylate was prepared in dimethyl sulfoxide (DMSO) and then diluted with RPMI 1640 media to a final DMSO concentration of 1%; this concentration of DMSO had no detectable effect on the leukemic cell growth. After treatment for 3 days, 100 μL of the CellTiter-Glo assay reagent was added to each well of the plates. After incubation for 10 min at 25° C., cellular luminescence was measured using a Tecan Infinite 200 microplate reader (Tecan Group, Switzerland). Results were expressed as percentages of the luminescence signal of analysed cells relative to untreated controls. To examine the synergism between HA-EGCG and FLT inhibitors, the combination index (CI) values were calculated based on the median-effect equation using the CompuSyn software (ComboSyn Inc., USA).

Results and Discussion

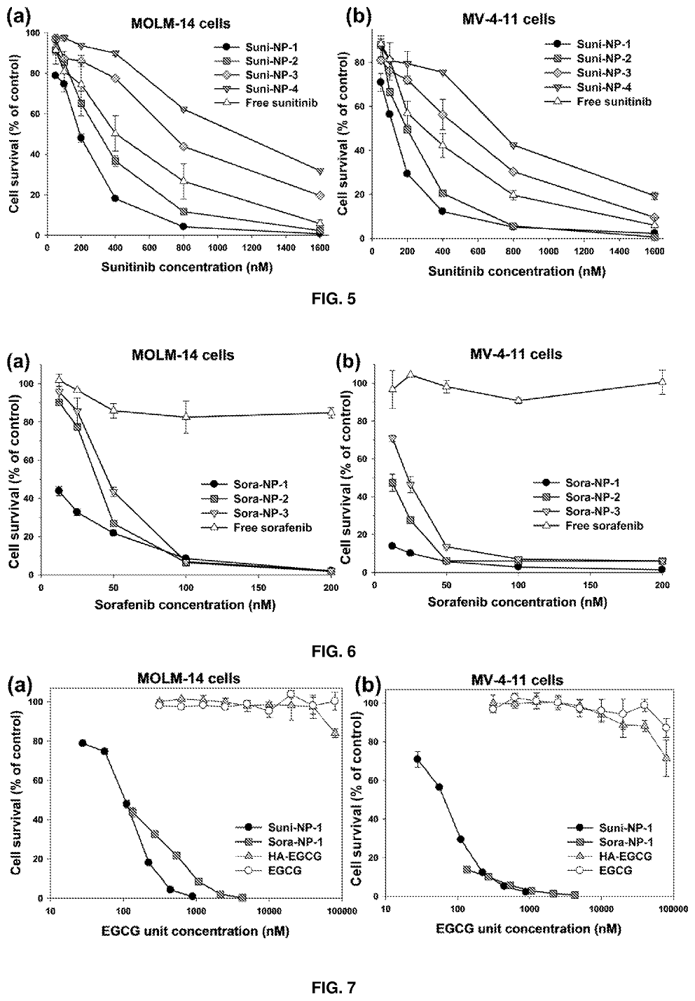

In vitro anti-leukemic activity of Suni-NP compositions were evaluated on two different FLT3-mutated AML cell lines: MOLM-14 cells (FIG. 5a) and MV-4-11 cells (FIG. 5b). The cells were treated for 3 days with Suni-NP or free sunitinib at equivalent concentrations, and their viability was analsed by the CellTiter-Glo assay that measures ATP content as an indicator of living cells. The order of effectiveness found in this study was Suni-NP-1>Suni-NP-2>free sunitinib>Suni-NP-3>Suni-NP-4. This was an unexpected result because it is generally known that nanoparticles with higher drug contents exhibit better therapeutic activity than those with lower drug contents.

In addition, it was also noted that the order of effectiveness was inversely correlated with the order of sunitinib loading content (Suni-NP-4 (46.9±0.47 wt %)>Suni-NP-3 (35.9±0.57 wt %)>Suni-NP-2 (8.89±0.19 wt %)>Suni-NP-1 (4.36±0.09 wt %); Table 1). Further, it appears that the particle size has insignificant impact on the in vitro anti-leukemic activity of Suni-NP compositions. For example, Suni-NP-2 showed stronger anti-leukemic activity than Suni-NP-4 although Suni-NP-2 had a larger particle size (164.8 nm) than Suni-NP-4 (142.3 nm). Considering that Suni-NP-1 having the strongest anti-leukemic activity had the highest HA-EGCG content (ca. 95.64 wt %) despite the lowest sunitinib content (ca. 4.36 wt %), it is reasonable to infer that Suni-NP compositions with higher HA-EGCG content tend to eradicate the leukemic blast cells more effectively than those with lower HA-EGCG content. These finding also implies that co-delivery of HA-EGCG and sunitinib at an optimal ratio would drive an enhancement of synergistic anti-leukemic effect.

The in vitro anti-leukemic activity of Sora-NP and free sorafenib were also evaluated on MOLM-14 cells (FIG. 6a) and MV-4-11 cells (FIG. 6b). All Sora-NP compositions were much more effective in killing the leukemic cells than free sorafenib at equivalent concentrations.

For example, treatment of all Sora-NP compositions at 200 nM eradicated over 99% of MOLM-14 cells, whereas the same dose of free sorafenib caused only a modest reduction (˜14%) of the cell viability. The order of effectiveness found in this study was Sora-NP-1>Sora-NP-2>Sora-NP-3>free sorafenib. The strongest anti-leukemic activity of Sora-NP-1 was likely ascribed to its smallest particle size, which is favorable for intracellular uptake, as well as the highest HA-EGCG content.

Next, the in vitro anti-leukemic activity of Suni-NP-1 and Sora-NP-1 was compared with that of HA-EGCG (C) conjugate and free EGCG. In this study, HA-EGCG (C) was selected because this conjugate was used to produce Suni-NP-1 and Sora-NP-1. For both MOLM-14 cells (FIG. 7a) and MV-4-11 cells (FIG. 7b), Suni-NP-1 and Sora-NP-1 induced more effective eradication of the leukemic cells than HA-EGCG conjugate as well as free EGCG, when compared at equivalent EGCG unit concentrations. This suggests a synergistic effect of HA-EGCG and FLT3 inhibitors on leukemic cell growth.

In addition, median-effect plot analysis was performed to investigate the synergistic effect of HA-EGCG and FLT3 inhibitors on the survival of MOLM-14 and MV-4-11 cells. Suni-NP-1 and Sora-NP-1 were chosen for this analysis because they were found to be the most effective among the tested nanoparticle compositions. The combination index (CI) values at various effective doses (ED₅₀, ED75, ED₉₀ and ED₉₅) were calculated based on the median-effect equation (Table 3). All the CI values for Sora-NP-1 were smaller than 0.1, representing very strong synergism between HA-EGCG (C) and sorafenib. This result proved that the synergistic anti-leukemic effect of HA-EGCG (C) and sorafenib was responsible for the observed superior potency of Sora-NP-1 over free sorafenib. Notably, Sora-NP-1 had markedly smaller CI values than Suni-NP-1, suggesting that the combined anti-leukemic effect of HA-EGCG (C) and sorafenib was much stronger than that of HA-EGCG (C) with sunitinib. Sora-NP-1 was selected for animal studies (in Example 4) because of its significant synergistic anti-leukemic activity.

TABLE 3
Combination index (CI) values for the combined effects of HA-EGCG and FLT3 inhibitors on
the survival of AML cell lines

| Sample | AML | Cl value at | Cl value at | Cl value at | Cl value at | |
|---|---|---|---|---|---|---|
| code | cell line | ED₅₀* | ED₇₅ | ED₉₀ | ED₉₅ | Level of synergy^{†} |
| Suni-NP-1 | MOLM-14 | 0.460 | 0.381 | 0.315 | 0.277 | Synergism |
| Suni-NP-1 | MV-4-11 | 0.401 | 0.394 | 0.388 | 0.384 | Synergism |
| Sora-NP-1 | MOLM-14 | 24.5 × 10⁻⁴ | 9.88 × 10⁻⁴ | 7.37 × 10⁻⁴ | 7.03 × 10⁻⁴ | Very strong synergism |
| Sora-NP-1 | MV-4-11 | 3.48 × 10⁻⁴ | 5.40 × 10⁻⁴ | 8.63 × 10⁻⁴ | 11.9 × 10⁻⁴ | Very strong synergism |

*EDx is defined as the dose of a drug required to cause x% inhibition of AML cell survival.
^{†}Level of synergy was determined from the Cl value at ED₅₀ based on the following criteria.
Very strong synergism Cl < 0.1; strong synergism: 0.1-0.3; synergism: 0.3-0.7; moderate synergism: 0.7-0.85; slight synergism: 0.85-0.90; nearly additive: 0.90-1.10.

### Example 4

### In Vivo Anti-Leukemic Activity of Selected HA-EGCG Nanoparticles Containing Sorafenib

To demonstrate the in vivo anti-leukemic activity of the nanoparticles of the current invention, HA-EGCG (C) nanoparticles containing sorafenib (denoted as Sora-NP-1) was further assessed on a pre-clinical patient-derived AML xenograft mouse model.

Experimental

All animal experiments were performed according to the protocols approved by IACUC at the Biological Resource Centre, Singapore. A pre-clinical patient-derived liquid xenograft mouse model was established based on a previous report (Z. Her, et al., J. Hematol. Oncol. 2017, 10, 162). Briefly, NOD-scid II2rg^{-/-} (NSG) newborn pups were sub-lethally irradiated at 1 Gy and engrafted with patient-derived AML cells named Leu 14. When the proportion of human CD45⁺ cells in peripheral blood reached around 10-15%, the mice were randomly divided into 3 groups. The first group received intravenous injections of isotonic dextrose solution containing Sora-NP-1 at a sorafenib dose of 0.4 mg kg⁻¹ twice weekly for 4 weeks. For comparison, another group of mice received intravenous injections of free sorafenib solution prepared in saline-DMSO mixture (95:5, v/v) at an equivalent dose. This concentration of DMSO was reported to cause no appreciable toxicity in mice (C. Carlo-Stella, et al., PLoS One 2013, 8, e61603). The last group did not receive any treatment and was used as a control. At selected time points, the proportion of human CD45⁺ cells in the peripheral blood, spleen and bone marrow was examined by flow cytometry analysis using a LSR II flow cytometer (BD Biosciences).

Results and Discussion

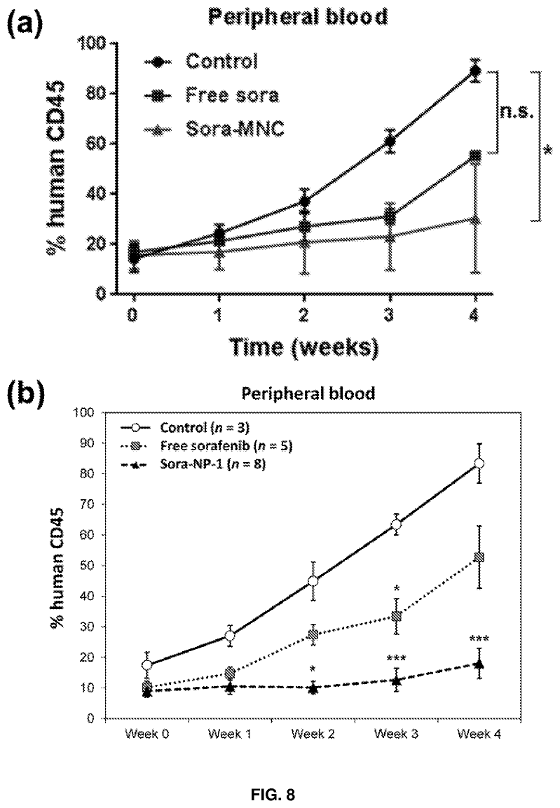

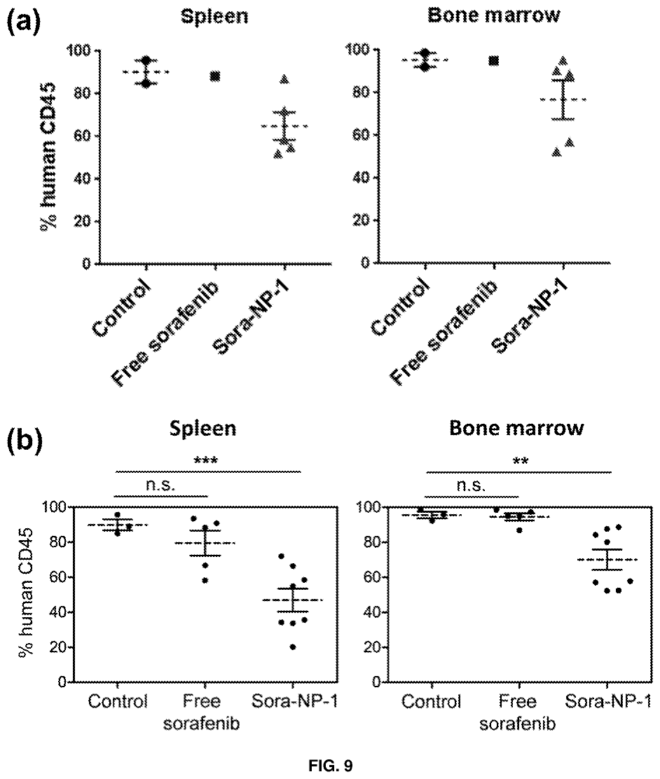

While Sora-NP-1 treatment induced a significant retardation (P<0.05) of AML cell proliferation in the peripheral blood when compared to the untreated control, only a slight delay of AML progression was observed from free sorafenib-treated group (FIG. 8a and b for initial and subsequent results, respectively). The enhanced systemic efficacy of Sora-NP-1 was probably attributed to its strong anti-leukemic activity observed in vitro and efficient internalisation by AML cells via HA-CD44 interactions. At the 4-week endpoint, the mice treated with Sora-NP-1 had a substantially lower proportion of AML cells in the spleen and bone marrow than those treated with free sorafenib at the same dose (FIG. 9a and b for initial and subsequent results, respectively). This result suggests that Sora-NP-1 enables more targeted delivery of sorafenib to the spleen and bone marrow than free sorafenib formulation, leading to more pronounced inhibition of AML cell propagation in those organs. The increased accumulation of Sora-NP-1 in the bone marrow and spleen would be beneficial for AML therapy because leukemic stem cells responsible for the relapse of AML are located primarily in the organs (D. S. Krause, et al., Nat. Med. 2006, 12, 1175-80). Neither body weight loss nor death was observed from the mice receiving Sora-NP-1 during the course of treatments, showing no sign of off-target toxicity.

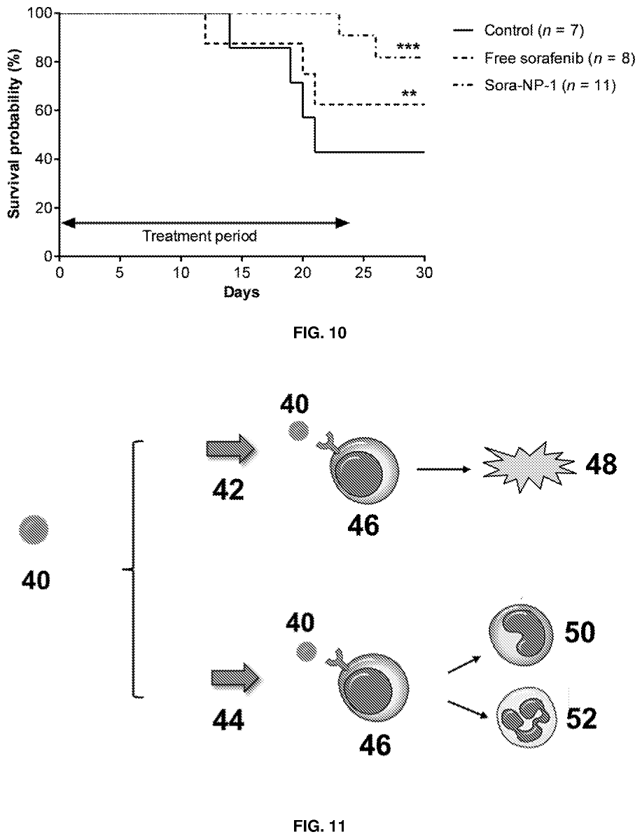

Kaplan-Meier analysis revealed that Sora-NP-1 treatment improved the survival of Leu 14-engrafted NSG mice more effectively than free sorafenib formulation (FIG. 10). While almost all the mice treated with Sora-NP-1 could survive during the treatment period (0-24 days), those treated with free sorafenib started to die earlier within 12-14 days probably due to the rapid AML progression. Considering that Sora-NP-1 treatment was halted on day 24, it would be possible to further extend the duration of survival through continued administration of Sora-NP-1. Collectively, the above results demonstrated the superior therapeutic efficacy of Sora-NP-1 over free sorafenib formulation in the patient-derived AML model.

### Example 5

### In Vitro Anti-Leukemic Activity of HA-EGCG (A) and (B) Conjugates

The therapeutic effects of HA-EGCG (A) and (B) (synthesised in accordance to General Synthesis 1 a and 2, M_{w} of HA=90 kDa) were evaluated on their in vitro efficacy in inducing differentiation and targeted-killing of AML cells. Two AML cell lines of different subtypes—HL60 (AML-M2) and NB4 (AML-M3) were used for the evaluation of the therapeutic potential of these HA-EGCG conjugates.

FIG. 11 shows a schematic representation of the strategy to use HA-EGCG (A) and (B) conjugates (40) in selectively targeting of AML cells 46 (i.e. myeloid blast cells) via HA binding to CD44 receptors overexpressed on the cell surface. Upon internalisation, HA-EGCG conjugates (40) can achieve anti-leukemic activity by a combination of two effects—elimination (42) of the blast cells by triggering cell death (48) of the blast cells, or induction of terminal differentiation (44) of the cells into monocytes (50) or granulocytes (52).

Experimental

Cell Lines

The human AML cell line HL60, human embryonic kidney cell line HEK293 and primary human umbilical vein endothelial (HUVEC) cells were purchased from the American Type Culture Collection (ATCC). The human AML cell lines NB4 was kindly donated by the Cancer Science Institute, Singapore. All the AML cells were cultured in RPMI 1640 medium supplemented with 10% fetal bovine serum (FBS) and maintained in density of 2×10⁵ to 1×10⁶ cells/mL. HEK293 cells were cultured in DMEM medium supplemented with 10% FBS while HUVEC cells were maintained in EBM™-2 Endothelial Cell Growth Basal Medium supplemented with endothelial cell growth medium SingleQuots™ supplements and growth factors. All the cells were maintained in a humid incubator with 5% CO₂ at 37° C.

Quantitative Assessment of CD44 Expression in AML Cells

To examine the CD44 expression levels, 5×10⁵ AML cells were suspended in PBS (pH 7.4) containing 0.2% (v/v) bovine serum albumin BSA and incubated with anti-human CD44 antibody or isotype control antibody (2 μg/mL) for 20 min at 4° C. (S. Ghaffari, et al., Leukemia 1996, 10, 1773-1781). The cells were then washed three times with ice-cold PBS containing 0.2% (v/v) BSA and then stained with FITC-tagged secondary antibody for another 20 min. Subsequently, the cells were washed again and analysed by flow cytometry using a fluorescence-activated cell sorter BD LSR II (BD Biosciences, Cailf.).

In Vitro Cell Viability Assay

All cells (HL60, NB4, HEK293 and HUVEC cells) were seeded at 1×10⁴ cells per 100 mL per well in 96-well plates. All AML cells were incubated for 2 h while HEK293 cells and HUVEC cells were allowed to attach overnight prior to treatment. Subsequently, the cells were treated with various concentrations of EGCG and HA-EGCG conjugates and incubated for a designated duration. After drug treatment, the cell viability was evaluated using the CellTiter-Glo™ Luminescent Cell Viability Assay Kit (Promega, Madison, Wis.) following the manufacturer's instructions. The luminescence from each well was measured using a Tecan Infinite microplate reader (Tecan Group, Switzerland). The final cell viability values were expressed as percentages derived from the luminescence intensity from the treated cells relative to untreated cells. All measurements were performed in triplicates.

Quantitative Evaluation of Differentiation in AML Cells

AML cells were seeded at 1×10⁵ cells in 1 mL per well in 24-well plates and were incubated for 2 h with 5% CO₂ at 37° C. The cells were then treated with HA-EGCG (A) at 500 μg/mL, HA-EGCG (B) at 250 μg/mL, together with HA and EGCG (38 μM) alone of equivalent concentrations. ATRA (1 mM), PMA (100 ng/mL) and A3D8 (0.6 μg/mL) were included as positive controls. After three days of incubation, the cells were harvested and examined for signs of differentiation by assessing cell surface antigen expression. To label the cell surface antigens, the cells were suspended in PBS (pH 7.4) containing 0.2% (v/v) BSA and then incubated at 4° C. for 30 min with mouse anti-human FITC-conjugated CD11b, Cy7-conjugated CD14 and Cy5-conjugated CD15 antibodies (2 μg/mL each). Mouse IgG1 isotype antibody was used as control. After that, the cells were washed three times with PBS containing 0.2% (v/v) BSA and the level of antibody binding was determined by flow cytometry using a fluorescence-activated cell sorter BD LSR II (BD Biosciences, Calif.). Each measurement comprised of acquisition of at least 1×10⁴ cells and the analyses were considered as informative when adequate numbers of events (>100) were collected in the enumeration gates. To quantify the percentage of antigen-expressing cells, the cells were defined to be positive for the antigens if they fell within the gating region pre-set to include <2% of untreated control cells.

Results and Discussion

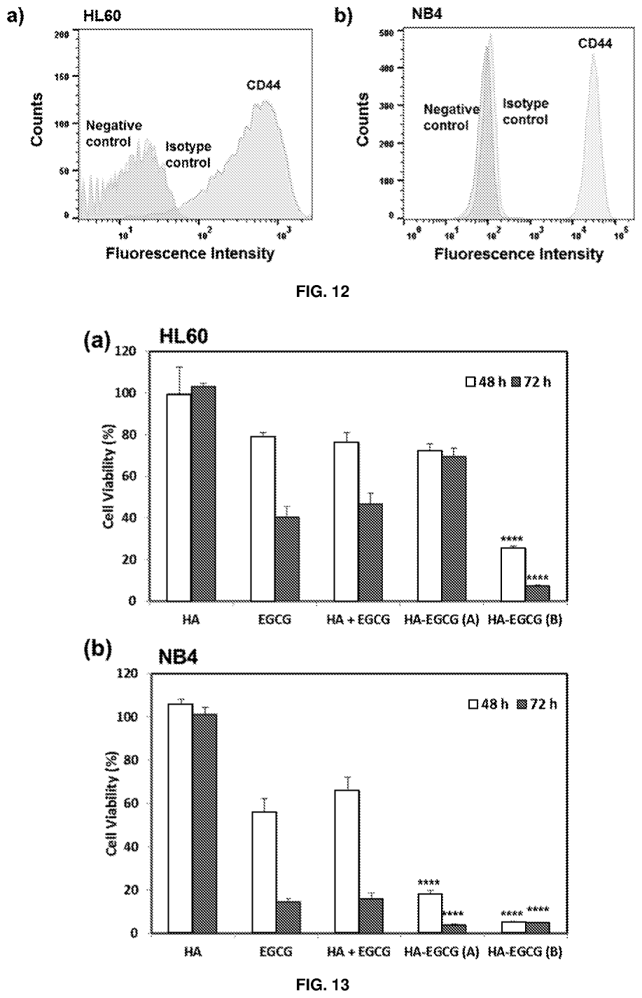

Firstly, the CD44 expression in both HL60 and NB4 cells was evaluated by flow cytometry, which confirmed the elevated levels of expression of CD44 in both AML cells (FIG. 12 and Table 4).

TABLE 4
Percentage of CD44-expressing cells in the two
AML cell lines of different subtypes.

| | | CD44- |
|---|---|---|
| Cell Line | AML Subtype | expressing cells (%) |
| HL60 | M2 | 95.3 |
| NB4 | M3 | 100 |

The viabilities of these cells were then assessed by incubating with HA-EGCG (100 μg/mL), HA, EGCG, and a mixture of HA and EGCG mixture at equivalent HA or EGCG concentrations, respectively. In HL60 cells, it was observed that HA-EGCG (B) demonstrated the highest toxicity among the five test groups in both AML cells (FIG. 13a), leading to the decline in the cell viability to 25.2±1.1% at 48 h and a further reduction to 7.3±0.3% at 72 h. No significant increase in toxicity was observed for HA-EGCG (A) treatment as compared to the treatment with EGCG, and a mixture of HA and EGCG, respectively. On the other hand, both HA-EGCG (A) and HA-EGCG (B) treatment led to significant greater toxicity in NB4 cells than EGCG, HA and a mixture of HA and EGCG at 48 h, reaching cell viability of 17.9±1.9% and 4.9±0.5% respectively (FIG. 13b). For HA-EGCG (A), the cell viability of NB4 was reduced to 3.9±0.4% with a longer incubation time of 72 h. In both cell types, EGCG alone and HA and EGCG mixture treatment resulted in similar toxicities while HA alone had limited effect on cell viabilities. The greater toxicity of HA-EGCG conjugates as compared to EGCG alone and HA and EGCG mixture could possibly be attributed to CD44 targeting of these AML cells facilitated by coupling of EGCG to HA.

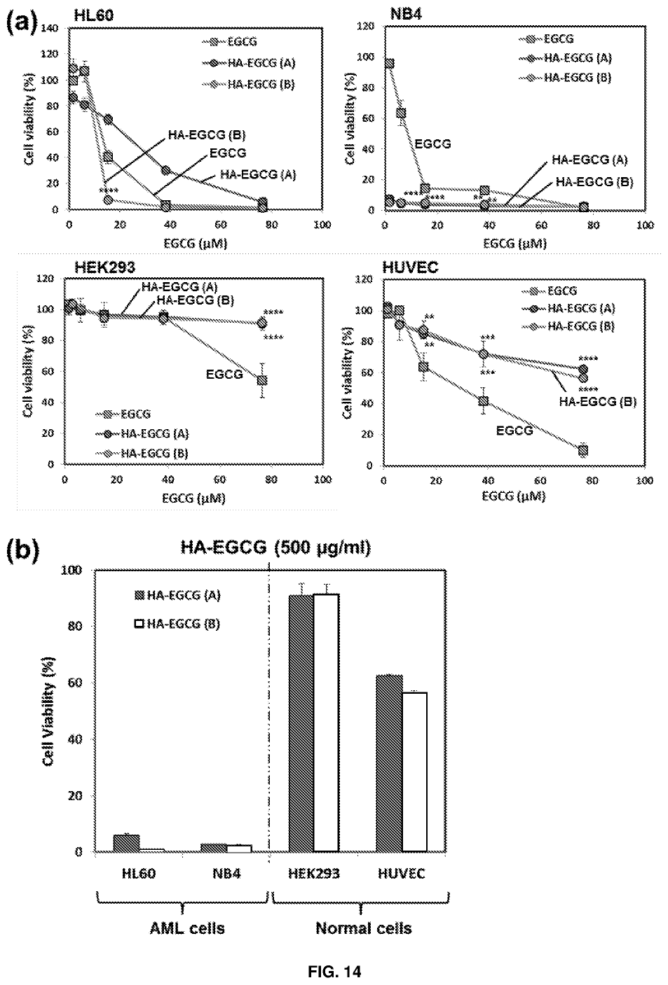

To assess the AML targeting specificity of HA-EGCG, the cytotoxicity of HA-EGCG (A) and (B) were evaluated on two normal cell types—human embryonic kidney cells (HEK293) and human umbilical vein endothelial cells (HUVEC). It was observed that increasing concentrations of both HA-EGCG (A) and (B), and EGCG alone led to a concomitant decline of the viabilities of all cell types after 72 h (FIG. 14a). Interestingly, HA-EGCG (B) demonstrated greater toxicity than EGCG in both AML cells with EGCG equivalent concentration range of 15-38 μM for HL60, and in the range of 1.5-76 μM for NB4. In contrast, EGCG demonstrated greater toxicity than HA-EGCG (B) in normal cells at EGCG equivalent concentration of 76 μM for HEK293 and in the range of 15-76 μM for HUVEC (FIG. 14a). Similarly, HA-EGCG (A) showed higher toxicity against NB4 cells as compared to normal HEK293 and HUVEC cells. Furthermore, HA-EGCG (A) and (B) at a fixed concentration of 500 μg/mL dramatically reduced the cell viabilities of AML cells by more than 94%. In contrast, 90.8±4.4% and 91.5±3.5% of HEK293 cells, and 62.5±0.6% and 56.5±0.8% of HUVEC cells remained viable upon treatment with HA-EGCG (A) and (B), respectively (FIG. 14b). Collectively, these results demonstrated that the toxicity of HA-EGCG was selective towards AML cells.

To evaluate the ability of HA-EGCG to induce terminal differentiation in AML cells, the expressions of three cell-surface antigens CD11b (common myeloid marker), CD14 (monocyte) and CD15 (granulocyte) in NB4 and HL60 cells after 72 h incubation with HA-EGCG conjugates were examined. Three previously reported differentiation-inducing agents (ATRA, phorbol 12-myristate 13-acetate (PMA) and anti-human CD44 antibody (clone: A3D8)) were also used as positive controls (T. R. Breitman, et al., Proc. Natl. Acad. Sol. U.S. A. 1980, 77, 2936-40; P. E. Newburger, et aL, Cancer Res. 1981, 41, 1861-1865; R. S. Charrad, et al., Nat. Med. 1999, 5, 669-676; R. S. Charrad, et al., Blood, 2002, 99, 290-299).

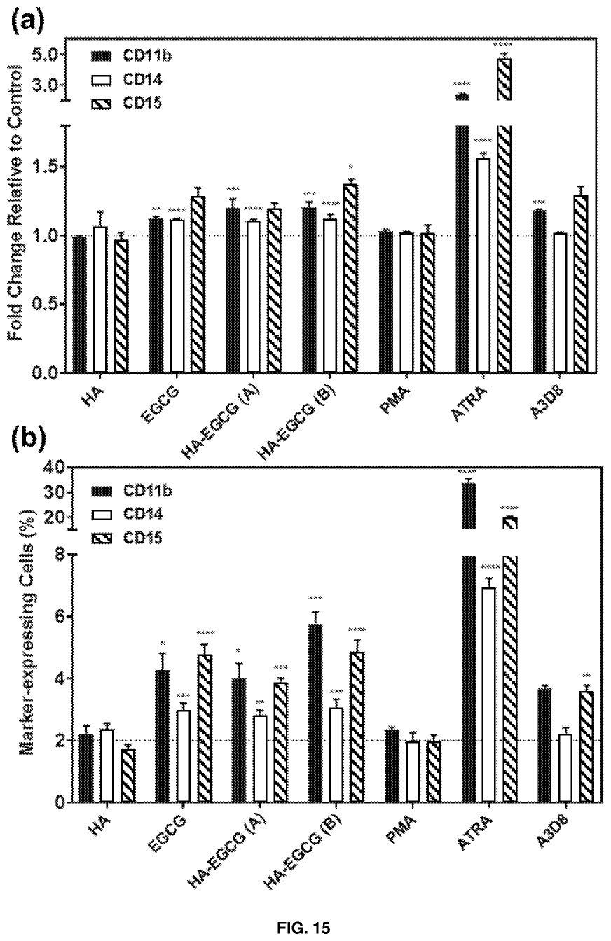

In NB4 cells, it was observed that the positive control ATRA gave the greatest increase in the three antigen expression levels and the proportion of antigen-expressing cells. HA-EGCG treatment significantly increased the expression levels of all three antigens ((CD11b: 1.2-fold, CD14:1.1-fold and CD15:1.2-fold for HA-EGCG (A); CD11b:1.2-fold, CD14:1.1-fold and CD15:1.4-fold for HA-EGCG (B) respectively) after three days (FIG. 15a). Furthermore, the percentage of cells expressing CD11b, CD14 and CD15 also showed significant increase with HA-EGCG (A) (CD11b:4.8%, CD14:2.8% and CD15:3.8%) and HA-EGCG (B) (CD11b:5.8%, CD14:3.0% and CD15: 4.9%) treatment (FIG. 15b). Similar effects were also noted in EGCG alone, but not in HA alone, which suggested that EGCG was mainly responsible for the induction of differentiation. Notably, HA-EGCG conjugates were superior to the other positive controls, PMA and A3D8, in promoting all three differentiation marker expressions. Among the three antigens, HA-EGCG increased CD11b and CD15 expressions to a greater extent than CD14, suggesting that HA-EGCG also supported preferential differentiation of NB4 cells to granulocytic lineage.

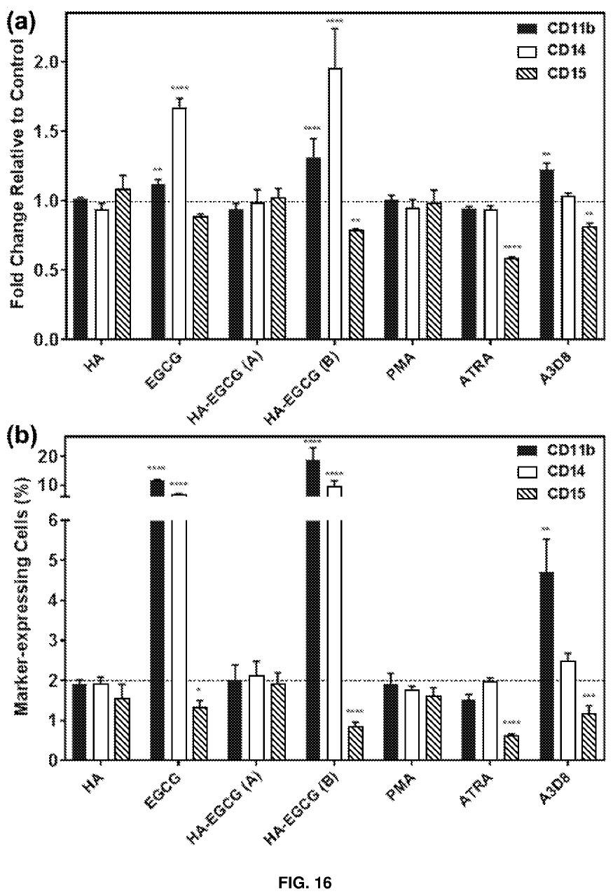

In HL60 cells, it was observed that HA-EGCG (B) treatment led to significant increases in the expression levels of CD11b and CD14 (CD11b: 1.3-fold and CD14: 2.0-fold) (FIG. 16a) and an increase in the percentage of HL60 cells expressing CD11b (18.6%) and CD14 (9.5%) (FIG. 16b). A significant reduction in the percentage of CD15-expressing HL60 cells was also noted. HA-EGCG (A) treatment did not lead to any enhancement in the expression of differentiation markers. Similar to NB4 cells, EGCG alone also showed similar trends as compared to HA-EGCG, confirming the major role of EGCG in inducing differentiation.

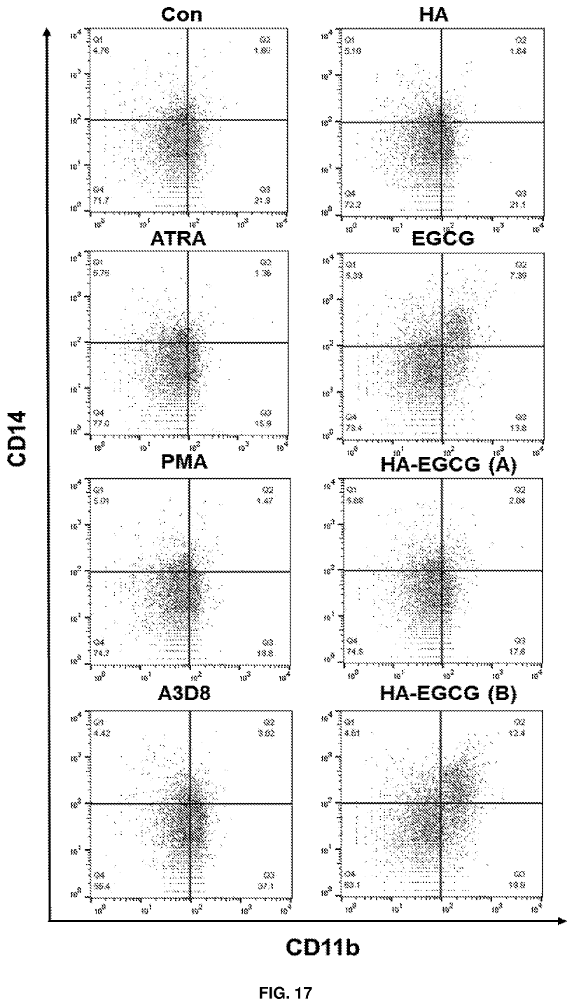

Further analysis of the HA-EGCG (A) and (B) treated HL60 cells revealed the distinct emergence of CD11b/CD14 double positive cell population in quadrant 2 (Q2) of the flow cytometry dot-plots (FIG. 17), which was absent in HL60 cells treated with all the positive controls—ATRA, PMA and A3D8. Interestingly, unlike the differentiation induction of NB4 cells towards CD11b/CD15 double-positive granulocytic lineage, this result supported the efficiency of HA-EGCG in enabling the differentiation of HL60 cells toward monocytic lineage. Collectively, these results provided clear evidence of the differentiation-inducing capability of HA-EGCG conjugates in both NB4 and HL60 cells.

### Example 6

### In Vivo Anti-Leukemic Activity of HA-EGCG (B) Conjugates

The in vivo anti-leukemic efficacy of HA-EGCG (B) conjugates (synthesised in accordance to General Synthesis 2, M_{w} of HA=90 kDa) was further assessed on a xenograft mice model of human AML HL60 cells.

Experimental

All animal experiments were performed in accordance to protocols approved by the Singapore Biological Resource Centre's Institutional Animal Care and Use Committee (IACUC). Anti-leukemic efficacy was evaluated using a previously developed AML xenograft model based on non-obese diabetic (NOD)/LtSz-severe combined immunodeficiency (SCID) IL2Rγ^{null} (NSG) mice (A. Agliano, et al., Int. J. Cancer 2008, 123, 2222-2227; E. Saland, et al., Blood Cancer J. 2015, 5, e297). NSG mice (6-8 weeks old) were irradiated with a sub-lethal dose of 2.5 Gy (60 cGy/min) from a photon radiation source 24 h prior to inoculation of 2×10⁶ HL60 cells via tail vein injections. The mice were then treated with intravenous injections (200 μL) of either sterile PBS as control or HA-EGCG (B) solution (50 mg/kg) three0 times weekly for a total of five weeks. To obtain hematopoietic cell counts, peripheral blood was collected by retro-orbital bleeding at designated time-points. Thirty microliter of blood was collected in heparin-coated tubes, which were subsequently analysed using a hematology counter (HEMAVET™ 950FS, Erba Diagnostic, Fla.). At the end of the study, the animals were sacrificed, and spleens were collected and weighed. The mice were monitored bi-weekly for symptoms of disease (scruffy fur, tumor-like lumps, weakness and reduced mobility) and all animals showing any signs of distress were euthanised.

Results and Discussion

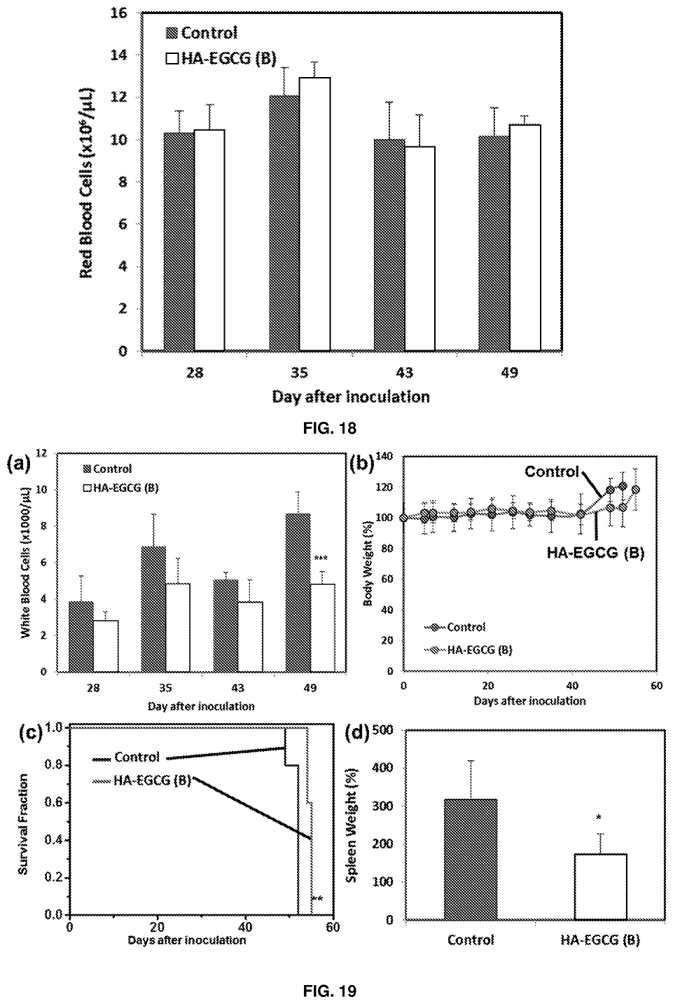

Two million HL60 cells were intravenously injected into sub-lethally irradiated (2.5 Gy) mice, which were subsequently treated with 50 mg/kg HA-EGCG (B) or PBS via tail vein injections every other day. The blood cell count was evaluated once a week one month post-injection and the survival of the mice was also monitored. While the red blood cell count was maintained around 10×10⁶ per μL in both the control and HA-EGCG (B) group (FIG. 18), the white blood cell count of the control mice showed a sharp increase from 5.0 to 8.7×10⁶ per μL at day 49 after injection (FIG. 19a). The white blood cell count was significantly lower in the HA-EGCG (B) treated mice (4.8×10⁶/μL), suggesting that HA-EGCG (B) delayed the onset of leukemia development. This is supported by the retardation in the body weight increase of the mice at day 49, possibly due to growth of cancerous lumps, as compared to the control (FIG. 19b). In addition, HA-EGCG (B) treatment prolonged the survival of leukemic mice (P<0.01) (FIG. 19c) and suppressed the dramatic increase (173% as compared to 317% of control group) in the weight of the spleen (FIG. 19d), a common characteristic of leukemic cell engraftment (A. Agliano, et al., Int. J. Cancer 2008, 123, 2222-2227; M. A. Papiez, et al., Food Chem. Toxicol. 2010, 48, 3391-3397). Taken together, these results demonstrated the efficacy of HA-EGCG (B) in the inhibition of AML progression in vivo.

1. A nanoparticle composition comprising:
nanoparticles formed from one of:
(a) a conjugate of dimeric epigallocatechin-3-O-gallate and hyaluronic acid, where the hyaluronic acid has multiple conjugation sites in its polymer backbone, where a plurality of dimeric epigallocatechin-3-O-gallate molecules are each conjugated to one of the multiple conjugation sites in the polymer backbone of hyaluronic acid;
(b) a conjugate of epigallocatechin-3-O-gallate and hyaluronic acid, where the hyaluronic acid has multiple conjugation sites in its polymer backbone, where a plurality of epigallocatechin-3-O-gallate molecules are each conjugated to one of the multiple conjugation sites in the polymer backbone of hyaluronic acid; or
(c) a epigallocatechin-3-O-gallate-terminated hyaluronic acid conjugate, where an epigallocatechin-3-O-gallate molecule is covalently bonded to a terminal position of the hyaluronic acid; and
an active agent or a pharmaceutically acceptable salt, solvate or prodrug thereof suitable to treat acute myeloid leukaemia, wherein:
the active agent is encapsulated in the nanoparticles.

2. The nanoparticle composition according to claim 1,
wherein:
(a) the conjugate of dimeric epigallocatechin-3-O-gallate and hyaluronic acid, where the hyaluronic acid has multiple conjugation sites in its polymer backbone, where a plurality of dimeric epigallocatechin-3-O-gallate molecules are each conjugated to one of the multiple conjugation sites in the polymer backbone of hyaluronic acid has a the formula Ia: 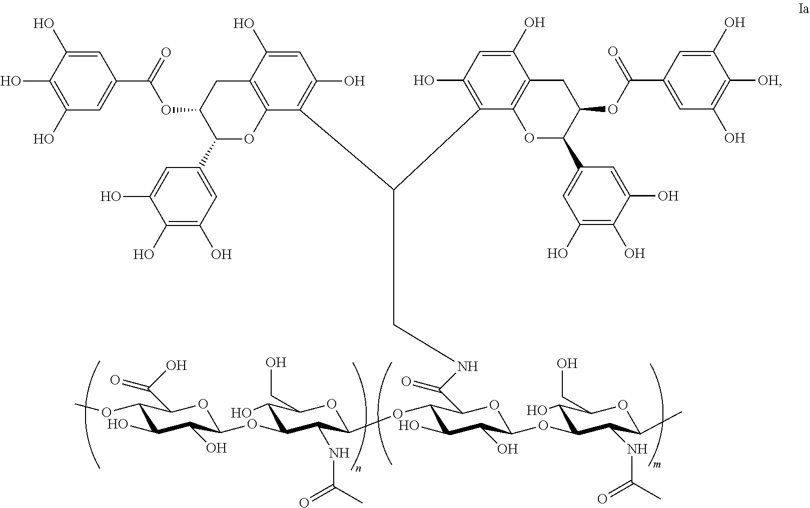
wherein each n and m represent random repeating units in the hyaluronic acid backbone; or
(b) the conjugate of epigallocatechin-3-O-gallate and hyaluronic acid, where the hyaluronic acid has multiple conjugation sites in its polymer backbone, where a plurality of epigallocatechin-3-O-gallate molecules are each conjugated to one of the multiple conjugation sites in the polymer backbone of hyaluronic acid has a formula Ib: 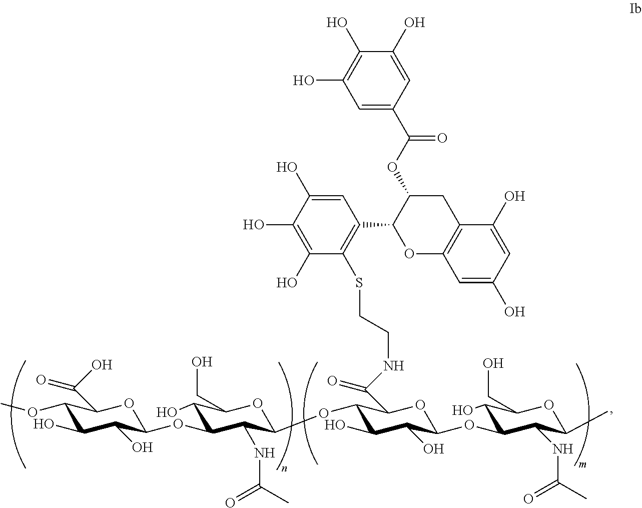
wherein each n and m represent random repeating units in the hyaluronic acid backbone; or
(c) the epigallocatechin-3-O-gallate-terminated hyaluronic acid conjugate has a formula Ic: 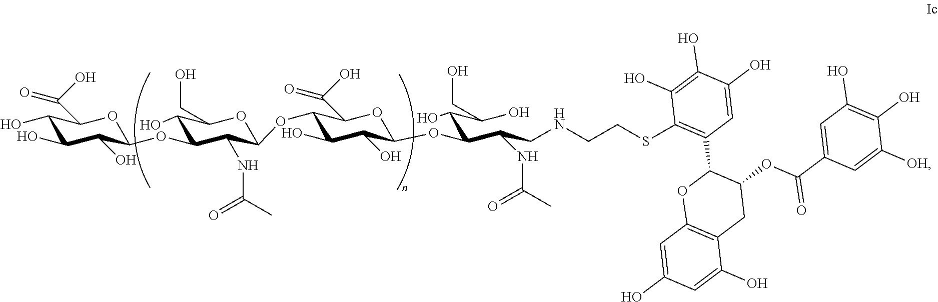
wherein n represent random repeating units in the hyaluronic acid backbone.

3. The nanoparticle composition according to claim 1, wherein:
(a) the epigallocatechin-3-O-gallate-terminated hyaluronic acid conjugate has a molecular weight of from 1 to 50 kDa;
(b) the conjugate of the epigallocatechin-3-O-gallate and hyaluronic acid where the hyaluronic acid has multiple conjugation sites in its polymer backbone, where a plurality of epigallocatechin-3-O-gallate molecules are each conjugated to one of the multiple conjugation sites in the polymer backbone of hyaluronic acid has a molecular weight of from 50 to 100 kDa; or
(c) the conjugate of dimeric epigallocatechin-3-O-gallate and hyaluronic acid where the hyaluronic acid has multiple conjugation sites in its polymer backbone, where a plurality of dimeric epigallocatechin-3-O-gallate molecules are each conjugated to one of the multiple conjugation sites in the polymer backbone of hyaluronic acid has a molecular weight of from 50 to 100 kDa.

4. The nanoparticle composition according to claim 1, wherein the nanoparticle has an average hydrodynamic diameter of from 10 to 1,000 nm.

5. The nanoparticle composition according to claim 1, wherein the active agent forms from 0.1 to 60 wt % of the composition.

6. The nanoparticle composition according to claim 1, wherein the active agent is an FMS-like tyrosine kinase receptor-3 (FLT3) inhibitor.

7. The nanoparticle composition according to claim 6, wherein the FLT3 inhibitor is:
(a) a Type I inhibitor;
(b) a Type II inhibitor.

8. The nanoparticle composition according to claim 7, wherein the FLT3 inhibitor is:
(a) sunitinib; or
(b) sorafenib.

9. The nanoparticle composition according to claim 1, wherein the nanoparticles of the epigallocatechin-3-O-gallate-terminated hyaluronic acid conjugate are core-shell nanoparticles:

10. A method of making a composition according to claim 1, wherein the method comprises:
(i) adding an active agent or a pharmaceutically acceptable salt, solvate or prodrug thereof suitable to treat acute myeloid leukaemia with one of:
(a) a conjugate of dimeric epigallocatechin-3-O-gallate and hyaluronic acid, where the hyaluronic acid has multiple conjugation sites in its polymer backbone, where a plurality of dimeric epigallocatechin-3-O-gallate molecules are each conjugated to one of the multiple conjugation sites in the polymer backbone of hyaluronic acid;
(b) a conjugate of epigallocatechin-3-O-gallate and hyaluronic acid, where the hyaluronic acid has multiple conjugation sites in its polymer backbone, where a plurality of epigallocatechin-3-O-gallate molecules are each conjugated to one of the multiple conjugation sites in the polymer backbone of hyaluronic acid; or
(c) a epigallocatechin-3-O-gallate-terminated hyaluronic acid conjugate, where an epigallocatechin-3-O-gallate molecule is covalently bonded to a terminal position of the hyaluronic acid,
in a solvent, optionally with agitation, for a period of time to provide a dispersion of nanoparticles; and
(ii) collecting the resulting nanoparticles from the dispersion of nanoparticles.

11. The method according to claim 10, wherein one or more of following applies:
(a) the solvent is water;
(b) a concentration of the active agent in solution is from 0.001 to 1 mg mL⁻¹; or
(c) a concentration of the conjugate in the solution is from 0.01 to 20 mg mL⁻¹.

12. A nanoparticle composition according to claim 1 for use in medicine.

13. (canceled)

14. A nanoparticle composition according to claim 1 for use in the treatment of acute myeloid leukemia.

15. A method of treatment of acute myeloid leukaemia comprising providing a pharmaceutically effective amount of the nanoparticle composition according to claim 1 to a subject in need thereof.

16. (canceled)

17. A compound of formula Ia or Ib: 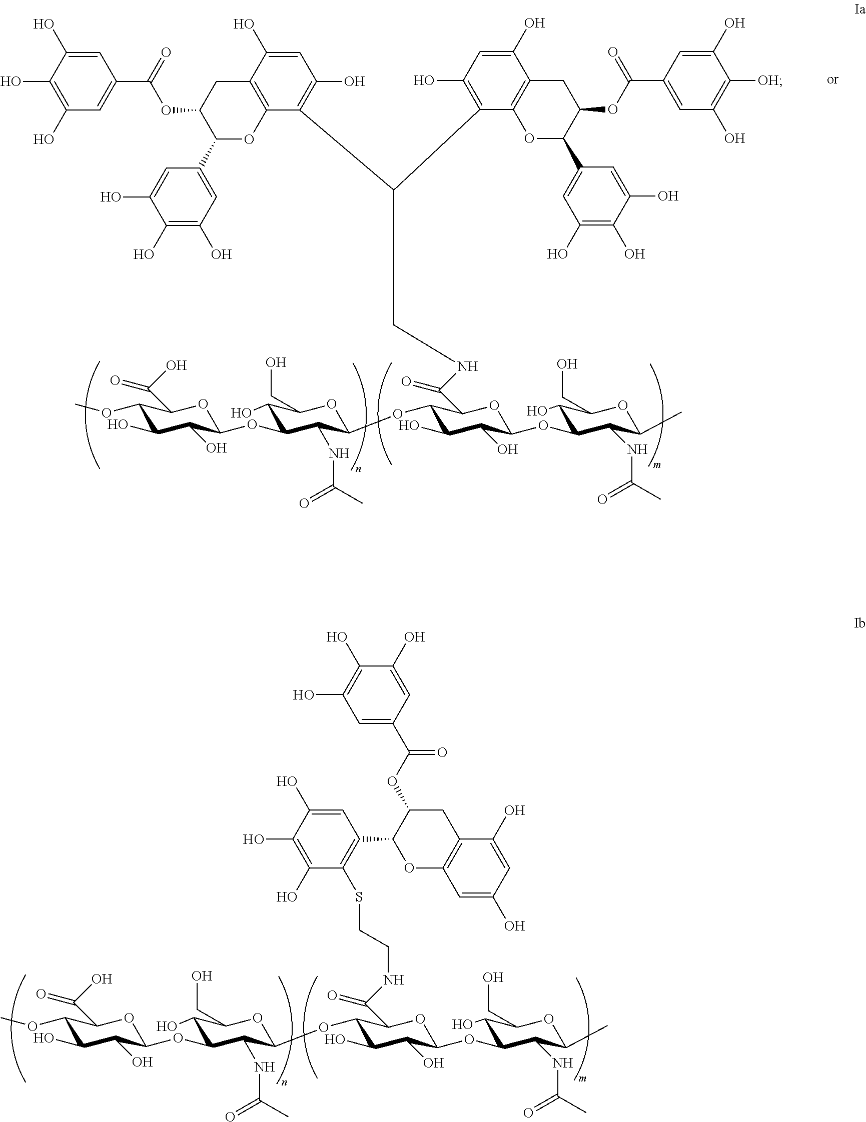
for use in treatment of cancer.

18. A method of treatment of cancer comprising providing a pharmaceutically effective amount of a composition compound of formula Ia or Ib: 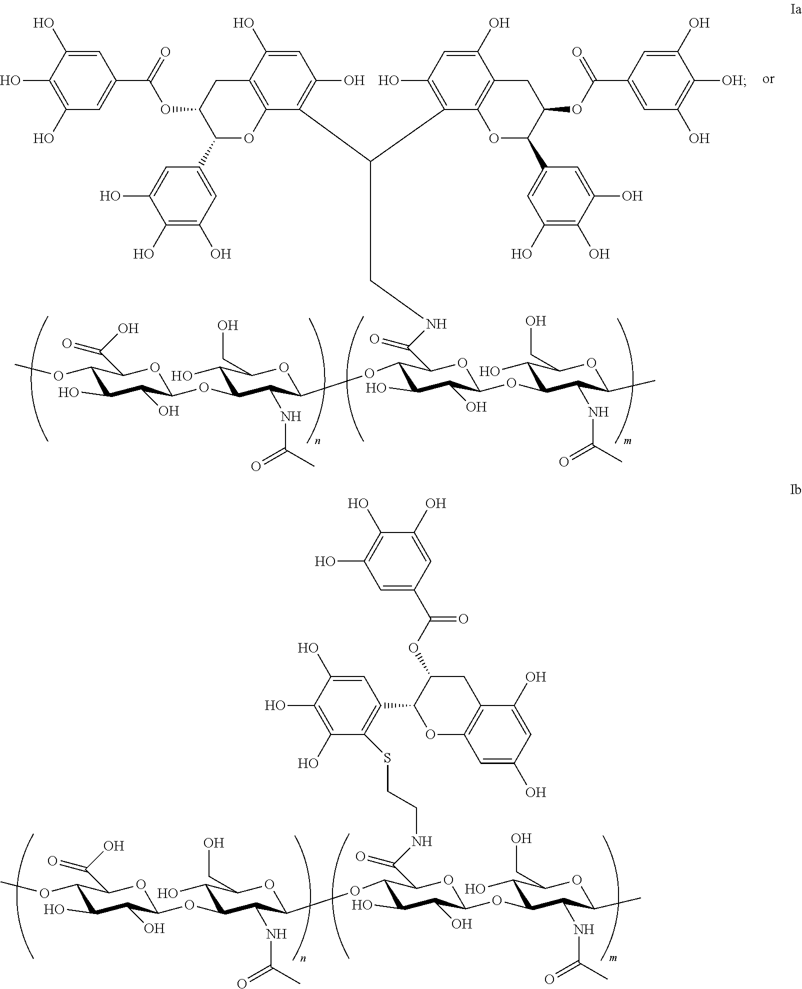
to a subject in need thereof.

19. The compound of claims 17, wherein cancer is acute myeloid leukemia.

20. The compound of claim 17, wherein:
(a) the compound of Ia has a molecular weight of from 50 to 120 kDa; or
(b) the compound of Ib has a molecular weight of from 50 to 120 kDa.

21. The nanoparticle composition according to claim 1, where the nanoparticles are formed from the conjugate of epigallocatechin-3-O-gallate and hyaluronic acid, where the hyaluronic acid has multiple conjugation sites in its polymer backbone, where a plurality of epigallocatechin-3-O-gallate molecules are each conjugated to one of the multiple conjugation sites in the polymer backbone of hyaluronic acid.

22. The nanoparticle composition according to claim 21, wherein the conjugate of epigallocatechin-3-O-gallate and hyaluronic acid, where the hyaluronic acid has multiple conjugation sites in its polymer backbone, where a plurality of epigallocatechin-3-O-gallate molecules are each conjugated to one of the multiple conjugation sites in the polymer backbone of hyaluronic acid has the formula Ib: 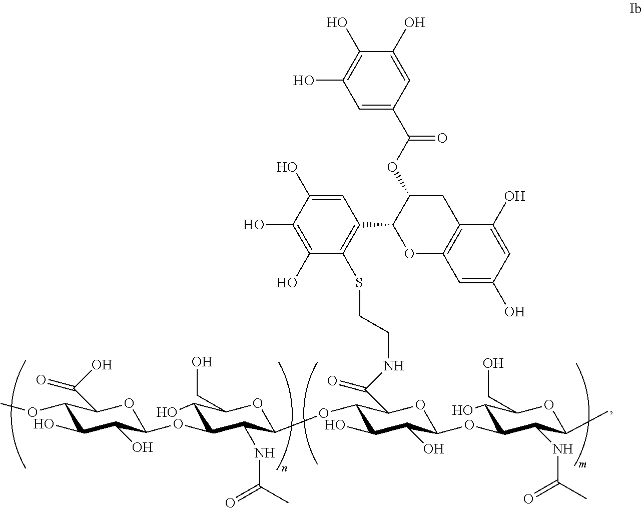
wherein each n and m represent random repeating units in the polymer backbone of hyaluronic acid.
